SEQUENCE LISTING (3 sequences)

SEQ ID NO: 1 - protein, 244 aa, Homo sapiens
MMRTQCLLGLRTFVAFAAKLWSFFIYLLRRQIRTVIQYQTVRYDILPLSPVSRNRLAQVKRKILVLDLDETLIHSHHDGVLRPTVRPGTPPDFILKVVIDKHPVRFFVHKRPHVDFFLEVVSQWYELVVFTASMEIYGSAVADKLDNSRSILKRRYYRQHCTLELGSYIKDLSVVHSDLSSIVILDNSPGAYRSHPDNAIPIKSWFSDPSDTALLNLLPMLDALRFTADVRSVLSRNLHQHRLW

SEQ ID NO: 2 - DNA, 1780 bp, Homo sapiens
gaaacccagctgcagaggctgcagccccggtccccagcggctaaaggacccccgagctcggggagggggaggccgctccggcccagcgctctgcgccctccggctccccctccccctccttccctgctccttcgctctgcaggtctcggaggaccccatcctagccctacctgtctcggcccgcaacctccccgaagccgtcggtgccactcccagcccatgtgggcccccgcgggctgcccacgcctgtcccccagctccccgttccgctgggctttaccctcgccaggggtggctttctgagccgcccgctccgtgcccctctctgcagcctctcctgccactcggggcccccgttccccctcccggcggcggggggctgcccccggggggctggcggagctgggccgcgggggccccggggccggcggtgccggggtcatcgggatgatgcggacgcagtgtctgctggggctgcgcacgttcgtggccttcgccgccaagctctggagcttcttcatttaccttctgcggaggcagatccgcacggtaattcagtaccaaactgttcgatatgatatcctccccttatctcctgtgtcccggaatcggctagcccaggtgaagaggaagatcctggtgctggatctggatgagacacttattcactcccaccatgatggggtcctgaggcccacagtccggcctggtacgcctcctgacttcatcctcaaggtggtaatagacaaacatcctgtccggttttttgtacataagaggccccatgtggatttcttcctggaagtggtgagccagtggtacgagctggtggtgtttacagcaagcatggagatctatggctctgctgtggcagataaactggacaatagcagaagcattcttaagaggagatattacagacagcactgcactttggagttgggcagctacatcaaggacctctctgtggtccacagtgacctctccagcattgtgatcctggataactccccaggggcttacaggagccatccagacaatgccatccccatcaaatcctggttcagtgaccccagcgacacagcccttctcaacctgctcccaatgctggatgccctcaggttcaccgctgatgttcgttccgtgctgagccgaaaccttcaccaacatcggctctggtgacagctgctccccctccacctgagttggggtgggggggaaagggagggcgagcccttgggatgccgtctgatgccctgtccaatgtgaggactgcctgggcagggtctgcccctcccacccctctctgccctgggagccctacactccacttggagtctggatggacacatgggccaggggctctgaagcagcctcactcttaacttcgtgttcacactccatggaaaccccagactgggacacaggcggaagcctaggagagccgaatcagtgtttgtgaagaggcaggactggccagagtgacagacatacggtgatccaggaggctcaaagagaagccaagtcagctttgttgtgatttgattttttttaaaaaactcttgtacaaaactgatctaattcttcactcctgctccaagggctgggctgtgggtgggatactgggattttgggccactggattttccctaaatttgtcccccctttactctccctctatttttctctccttagactccctcagacctgtaaccagctttgtgtcttttttccttttctctcttttaaaccatgcattataactttgaaaccaaagcgg

SEQ ID NO: 3 - protein, 6 aa, Homo sapiens
KVVIDK

(19) United States
(12) Patent Application Publication
(10) Pub. No.: US 2015/0004621 A1
(43) Pub. Date: Jan. 1, 2015

(54) BIOLOGICAL MARKER FOR EARLY CANCER DETECTION AND METHODS FOR CANCER DETECTION (BF819)

(71) Applicant: MILAGEN, INC., Emeryville, CA (US)

(72) Inventor: Moncef Jendoubi, San Francisco, CA (US)

(21) Appl. No.: 13/931,432

(22) Filed: Jun. 28, 2013

(51) Int. Cl. G01N 33/68

(52) U.S. Cl. CPC G01N 33/6893
USPC 435/7.1; 436/501

(57) ABSTRACT

BF819 is a biomarker for the early detection of cancer. The natural polypeptide sequence of BF819 is disclosed along with the sequence of an epitope bound by a novel mAb BF819 used in tests and methods for cancer detection. Specific cancer and tumor types are identified where BF819 is overexpressed along with data showing the extent of the detection of BF819 in cancer, normal, and benign conditions.

### BACKGROUND

Over a million and a half estimated new cancer cases (1,638,910) in the US in 2012 caused over half a million (577,190) deaths. Over a lifetime, roughly half of all people between the ages of 50 to 70 will get some form of cancer. Cancer is the second leading cause of death after heart disease. The overall cost of cancer treatment exceeds half a trillion dollars and is constantly increasing.

The four major cancers in the US are breast, prostate, lung and colorectal (Siegel R et al., Cancer statistics, 2012, CA Cancer J Clin 62:10-29, 2012). In terms of mortality, ovarian and pancreatic cancers are the most deadly accounting for 6 and 7% respectively of cancer estimated deaths in 2012, while representing only 3% of all cancers diagnosed. Id.

Pancreatic cancer, while representing only 6% of estimated new cancer cases in 2012, is responsible for 11% of cancer deaths (Siegel 2012). In the early stage, pancreatic cancer is a relatively symptomless disease. Patients usually present at an advanced stage and only 10-15% of patients have small resectable cancers (ACS, 2012) that are candidates for surgery. For all stages combined, the 1-year relative survival rate for pancreatic cancer is 24%, and the 5-year rate is about 4%. Id. Pancreatic cancer has the shortest life expectancy of all malignancies after discovery, with a median survival rate of −18 months.

Kidney cancer represents 8% of all cancers in the US, with 64,770 estimated new cancer cases and 13,570 deaths in 2012. Id. Renal carcinoma represents 92% of all kidney cancers, and is mostly asymptomatic at early stage. Risk factors include heavy smoking, obesity, hypertension and occupational exposure to certain chemicals. Kidney cancer tends to be resistant to traditional chemotherapy and radiation therapy treatments and no reliable screening test exists (ACS, 2012).

One of the most important factors affecting the survival rate of all cancers is early detection. For many cancers, detection at the earliest stages yields survival rates greater than 90%, while detection at the later stages often causes survival rates to fall below 10%. In most cases, cancer is not detected until a proliferation of cancer cells is physically quite large, such as when an excess growth of tissue creates a lump or other mass that can be seen or felt by a cancer patient or when this mass causes pain or altered function in surrounding tissues or organs.

However, the earliest stages of cancer cause profound changes in the basic physiology of a patient, including changes at the genetic level. While excess cell growth itself causes fundamental changes, other physiological mechanisms are also affected when the cancer grows and spreads throughout the body. Changes in a cancer patients' DNA such as chromosomal alterations, alterations in gene sequences, and altered gene expression patterns also lead to modifications in protein expression. These changes in protein expression at the cellular level correlate with subtle changes in organs, tissues, and body fluids.

Although it is well recognized that a large number of proteins that are involved in the onset and development of cancer are fundamentally altered in terms of their structure, function, or expression, scientists have had limited success in identifying specific proteins that are uniquely associated with the development of cancer and are not found in normal patients. If such proteins could be reliably identified, detection of the proteins would be a valuable tool for the early detection of cancer leading to increased cancer survival rates in the entire population.

Where a particular protein is expressed only in cancer patients, or is expressed in a unique chemical form, or has any other distinguishing feature that distinguishes normal from cancer patients, such a compound is called a “cancer marker.” For many years, doctors and scientists have searched for cancer markers that uniquely identify the earliest onset of cancer. Ideally, these markers would not be present in other diseases or in benign conditions such that detection of such a marker would provide a reliable indicator that patient was in the earliest stages of developing cancer. In addition to early detection, these markers could be used to determine a prognosis in a patient, to monitor disease progression, or to predict a patient's response to surgery or chemotherapy.

While several potential markers have been analyzed for early cancer detection, very few have actually reached the clinical setting. Recommendations for a number of cancer markers have recently been reviewed by the National Academy of Clinical Biochemistry (NACB) and the American Society of Clinical Oncology (ASCO) panels: in breast cancer (Duffy, 2009; Harris, 2007), colon cancer (Brunner, 2009), lung cancer (Stieber, 2006), prostate cancer (Lilja, 2009), pancreatic cancer (Goggins 2005; Locker, 2006; Duffy, 2010), ovarian cancer (Chan, 2009), and cervical cancer (Gaarenstroom, 2007). A great need remains for early detection cancer markers because many existing markers, such as CEA, CA-15, CA-19, and CA-125, are elevated only in advanced cancer stages. In colon cancer, no effective early stage biomarkers exist, whether tissue or serum-based. While there are methods available for early detection and screening for colon cancer, such as FOBT and colonoscopy, FOBT has limited sensitivity and the latter is an invasive procedure, resulting in only 44% of US adults over the age of 50 undergoing screening (ACS, 2012). No lung cancer or ovarian cancer early detection screening technique is currently available (Stieber, 2006; Smith, 2008). Like many cancers, ovarian cancer is a rather symptomless disease at the early stages, and is mostly detected at advanced stage with imaging and serum CA-125 marker measurements (Chan, 2009), at which point aggressive treatments such as surgery or chemotherapy are less likely to be successful.

PSA screening for prostate cancer in men age 45-50 has been the early detection gold standard for the past few decades (Smith, 2008; Lilja, 2009). However, it is now recommended that patients be informed of the pros and cons of PSA testing prior to screening (ACS, 2012). Where a candidate marker does not adequately distinguish cancer patients from normal patients, for example incorrectly indicating the risk of cancer in patients that are entirely normal, or where the marker fails to detect cancer in a patient, the costs of a misdiagnosis can vastly outweigh the benefits. The limitations of PSA as an early detection marker emphasizes the need for new and better stand-alone biomarkers, or additional biomarkers to supplement and improve current ones.

Some tests have shown an ability to predict whether a tumor in a patient is particularly aggressive. However, these tests typically require a tissue sample taken by an invasive procedure, such as a biopsy from the tumor, for gene expression analysis. These tests are not capable, or practical, for use in early detection in patients having no current symptoms.

Moreover, where the performance of the marker in separating cancer from normal is not adequate, the marker would have no utility when applied to the general population. In other words, while a marker may be used in patients already diagnosed with cancer, or in those at high risk, the ideal marker would be able to reliably distinguish a normal patient from an early cancer patient with enough accuracy that the marker could be used to screen the generally healthy population for early detection of cancer.

Furthermore, while scientists who analyze cancer tissue can readily detect fundamental differences between tumor tissue and regular tissue, those differences are not always attributable to the cancer itself and may be the result of inflammation or other events or conditions that are not directly related to the early onset of cancer. Furthermore, the examination of cancer tissue is not a viable approach for the early detection of cancer in the general population. It is simply impractical, and would be overly burdensome and costly, to surgically remove tissue samples from the general population, even in those patients where a high risk of a tumor exists. Furthermore, the methods to detect cancer often involve expensive and potentially damaging analytical methods, such as x-rays and CT scans, that cannot be routinely applied to the population at large and are reserved for only those cases where a clinical diagnosis is already made.

Therefore, an ideal cancer marker would satisfy several different criteria: 1) the marker would identify the onset of cancer at an early stage where the prognosis for a cure and long-term survival are the greatest, 2) the marker would distinguish between normal patients, or those with a benign condition, and early stage cancer patients with very high reliability and would yield limited false negative results, i.e. failing to detect the early development of cancer in patients who in fact have an early stage cancer, and would yield limited false positives, i.e. incorrectly identifying a patient with cancer who is actually cancer free.

Still further, an ideal marker for the early detection of cancer would be simple and inexpensive to detect and could be detected in a patient's body fluid such as blood or urine, such that the test could be performed without a biopsy to remove tissue or other invasive or expensive procedures. Also, an ideal marker could be measured as a simple laboratory test that is conveniently and routinely performed as part of a regular visit to the doctor.

Because a wide variety of blood tests and urinalysis are routinely performed in doctors' offices and medical laboratories, a test kit or method for the early detection of cancer would be a powerful addition to the existing battery of tests performed on patients as part of ordinary health management. Moreover, in patients who are at high risk of developing cancer, i.e. certain patients in the aging population or with a family history or other history indicating a high risk of cancer, the ability to detect and treat cancer at the earliest stages would save millions of lives and preserve billions of dollars in resources otherwise dedicated to treating late stage cancer.

Therefore, an urgent need exists for cancer markers for all types of cancer where the marker enables non-invasive early cancer detection methods, and where tests identifying the marker are accurate, reliable, sensitive and specific, and that can be applied to the asymptomatic general population. If such markers were identified, they could also be used to obtain a prognosis upon detection in the body, to track the progression or metastasis of cancer and to track the treatment response once surgical or drug therapy begins.

### SUMMARY OF THE INVENTION

The core of this invention is compositions and methods related to a protein cancer marker for the early detection of cancer and new applications of knowledge about this marker, the gene(s) and synthetic gene constructs encoding the marker, newly created antibodies to the marker, and complexes at the naturally occurring marker and the monoclonal antibodies whose characteristics, epitope, and creation are described below. The protein marker itself has the amino acid sequence identified below, along with the DNA encoding the polypeptide sequence of the marker. A novel monoclonal antibody (mAb) capable of binding the polypeptide is disclosed, together with a defined epitope at which binding to the marker takes place. A sequence listing is submitted herewith containing the amino acid sequence of the marker (SEQ ID NO:1), the polynucleotide sequence of the gene for the marker (SEQ ID NO: 2), and the amino acid sequence of the epitope at which the mAb binds (SEQ ID NO: 3). The antibody may also contain markers or other functional entities allowing for the detection or localization of the marker, or the mAb, as well as for the detection of the binding of the antibody to the marker to form a complex at the epitope. The detection of the marker, the antibody, the gene or related species such as pre-RNA, mRNA, etc. can take place in an in vitro diagnostic kit for detection of cancer in a biological sample, or in a patient test sample, and in a large scale, high throughput format assay method or system for processing large numbers of samples.

The detection also includes detecting non-natural variants of each of the foregoing in any assay format. The format for detection of the protein marker is not critical to utility of the invention and the marker and related species as defined herein can be detected by any existing technique for accurate identification of a polypeptide or polynucleotide sequence, or synthetic constructs based thereon, in a biological or patient sample.

Because the protein marker is secreted from the cells of a human patient into a “biological fluid” or “patient test sample”, typically the blood or urine of the patient, the detection of the marker using conventional assay platforms for analysis of blood and urine is included within the invention. Identification of the marker also enables the detection of autoantibodies where present. The antibodies described below for binding the marker may be used in any laboratory test format that uses a binding reaction between the polypeptide marker and the antibody to determine the presence of the marker in a biological sample. Also, based on the identity of the epitope for binding of the mAb, additional methods for using other mAbs specific for the marker or other techniques to detect the epitope in variants of the marker are enabled.

The invention also includes methods for detecting the polynucleotide, downstream transcripts of the polynucleotide, pre-RNA, mRNA, or any species associated with transcription of the polynucleotide disclosed herein or any species associated with the translation process yielding the marker. Also, the polypeptide marker itself, may be transformed into a derivative or synthetic construct useful for detection or creating antibodies for detection of the marker or a variant thereof. Novel reagent grade monoclonal antibodies to the polypeptide marker are provided, including an identification of the specific epitope at which the mAb binds to the marker. The methods of the invention include measurement or detection of any component of the polypeptide marker including fragments, modifications, post translational modifications, truncations, or essentially any adequate representative sample of an amino acid sequence of which the polypeptide marker is comprised to determine the presence of the polypeptide in a sample. This includes using novel mAbs enabled by the description below to separate the marker described herein from a biological sample, such as a patient test sample in a test format wherein secreted proteins are identified. The mAbs described herein can also be used in a diagnostic method to manufacture a new composition comprised of a complex of the novel mAb and the marker. The methods also include distinguishing expression or secretion of the marker from other isoforms or variants of the marker, particularly where the detection events indicate the presence or progression of cancer or prognosis for, or response to treatment.

Specific uses of the methods described herein include detection of early cancer in the asymptomatic general population, detecting cancer in a suspect patient population having a high risk of developing cancer, tracking the status or progression of cancer in a patient, including the efficacy or success of a course of treatment over time by sequential measurement of the marker in a patient, preferably by secretion into a body fluid, but also including through measurement or analysis of gene expression or in tissue marker detection following a biopsy or imaging event. Similarly, by tracking the marker across a single patient over time, or through a population of patients at a fixed point in time or across numerous time periods, the efficacy of a new cancer treatment may be assessed. For example, where a new cancer therapeutic compound is under investigation, sequential measurements of the presence or quantity of the marker in a patient or a patient population provides an indication of the therapeutic utility of the clinical candidate.

The invention also includes test devices, kits or methods for detecting the marker or related species, either alone or in combination with other markers, to assess the health or condition of a patient. The test can be in a panel format including the polypeptide and portions thereof, the polynucleotide, antibodies, or other entities or constructs described herein. The invention includes compositions specifically formulated and constructed for use as imaging agents to detect and localize the presence of the marker, or a form or variant thereof, in tissue or in an organ in the human body. Imaging or detection of the marker in vivo may include or be followed by biopsy, target radiation, or chemical therapy when or where the marker is detected.

The methods of the invention include the techniques and protocols specifically used for testing the asymptomatic general patient population for cancer, diagnosing a patient or groups of patients, and the practice of predictive medicine, including where specific populations of patients are identified and tested for the early development of cancer. These specific or pre-determined populations can be defined by age, sex, ethnic origin, prior disease, family history, genetic markers (such as Her-2, BRCA 1/2), exposure to toxins, carcinogens, or environmental or other cancer risk factors, or any event that places a patient in a defined or higher risk population.

The invention provides methods of determining or predicting effectiveness or response to a particular treatment, and methods of selecting a cancer treatment for an individual. For example, markers that are differentially expressed by cells (e.g., cancer cells) that are more or less responsive (sensitive) or resistant to a particular cancer treatment are useful for determining or predicting effectiveness or response to the treatment or for selecting a treatment for an individual.

Finally, the invention includes methods to detect cancer in an individual by measuring specific amounts of circulating or secreted marker in a biological or patient test fluid, such as in urine serum, by immunological or other methods.

### DESCRIPTION OF FIGURES AND TABLES

FIG. 1: Epitope and amino acid sequence of BF819

Amino acid sequence of BF819. The epitope sequence recognized by mAb BF819 is underlined.

FIG. 2: Nucleotide sequence of BF819

Nucleotide sequence of BF819 cDNA. Coding sequence spans nucleotides 448 to 1182.

FIG. 3: Differential expression of BF819 in cancer versus normal tissues by MPAT

Protein extracts prepared from normal, normal adjacent tumor (NAT), benign, and cancer samples from breast, colon, lung and ovary tissue specimens were prepared according to Example 4, spotted in the same amount on the matrix protein array, and overlaid with mAb BF819. Immunodetection was via the fluorescence-based Li-cor Odyssey detection system as detailed in Example 3.

The figure shows the colon, lung and ovary section of the membrane from the 1329 sample experiment (See Table 1). Spot position is indicated via a letter (row) and a number (column). Each section features 48 samples per row. In the colon section, samples are located as follows: colon NAT: A1-E48 (240), colon benign: F1-F23 (23, with F24-F48 empty), early stage colon cancer: G1-H41 (89; with H42-H48 empty), late stage colon cancer: I1-J36 (84, with J37-J48 empty). In the lung section, samples are: lung NAT: A1-C16 (112, with C17-C33 empty), lung benign: C34-C48 (15), early stage lung cancer: D1-G12 (156, with G13-G48 empty); late stage lung cancer: H1-H43 (43, with H44-H48 empty). In the ovary section, samples are located as follows: ovary NAT: A1-A43 (43, with A44-A48 empty), ovary benign: B1-B35 (35, with B36-B48 empty), early stage ovary cancer: C1-C36 (36, with C37-C44 empty), and late stage ovary cancer: C45-D48 (52). Table 1 lists sample numbers in each category and at each organ site.

FIG. 4: CEA detection in cancer tissues by MPAT

Protein extracts prepared from normal, normal adjacent tumor (NAT), benign, and cancer samples from breast, colon, lung and ovary tissue specimens were prepared according to Example 4, spotted in the same amount on the matrix protein array, and overlaid with a commercial mAb against CEA. Immunodetection was via the fluorescence-based Li-cor Odyssey detection system as detailed in Example 3.

The Figure illustrates CEA immunodetection in breast, colon, lung and ovary tissue extracts from the 1471 sample experiment (Table 1). Spot position is indicated via a letter (row) and a number (column). Each row features 48 samples.

In the colon section, samples are located as follows: colon NAT: A1-F44 (284, with F45-F48 empty), colon benign: G1-G17 (17), early stage colon cancer: G18-116 (95), and late stage colon cancer: 117-K23 (103, with K24-K48 empty). In the breast section, samples are: breast NAT: A1-C44 (138, with B35, B41, C45-C48 empty), breast benign: D1-D22 (22), early stage breast cancer: D23-E26 (49, with E4, E14, E24 empty), and late stage breast cancer: E27-F45 (67). In the lung section, samples are: lung NAT: A1-E41 (233), lung benign: E42-F7 (14), early stage lung cancer: F8-14 (141) and late stage lung cancer: 15-148 (44). In the ovary section samples are: ovary NAT: A2-B35 (78, with A5, A8, A16, A20 empty), ovary benign: B36-D1 (62), early stage ovary cancer: D2-D37 (36), and late stage ovary cancer: D38-F30 (87, with E11, E19, F31-F48 empty). Table 1 lists sample numbers in each category and at each organ site.

FIG. 5: Immunodetection of BF819 expression in 28 cancer cell lines by MPAT

Panel A: Diagram illustrating the spot location of 28 cancer cell line protein extracts in the MPAT assay. Cancer cell lines are identified by numbers, while their name and tissue of origin are indicated below the diagram. Equal amounts of protein extracts from each cancer cell line are spotted on the MPAT membrane in duplicate.

Panel B: Marker expression in cancer cell lines via MPAT immunodetection, as described in Example 5, using the fluorescence-based Li-cor Odyssey detection system, as described in Example 3. Spot intensity relates to expression levels.

FIG. 6: Western blot analysis of BF819

Immunodetection of BF819 with mAb BF819 by Western blot using total protein extracts prepared from different cancer cell lines, and mixed as indicated. Lane C/B: colon cancer (WiDr) and breast cancer (MDA-MB 231) cell lines; lane P/L: prostate cancer (DU 145) and lung cancer (NCI-H1792); lane 0/L: ovary cancer (ES2) and lung cancer (NCI-H157).

As described in Example 7 ten microgram of protein extracts are separated on a SDS-PAGE and transferred to a nitrocellulose membrane, followed by incubation with mAb BF819 and immunodetection. Arrows point to major protein bands detected according to a ladder of protein molecular weight standards.

FIG. 7: Immunodetection of secreted BF819 from cancer cell lines

Left panel: Diagram illustrating the spot location of 12 cancer cell lines in the MPAT assay. Equal amounts of proteins extracts from the tissue culture supernatants of each cell line are spotted, in duplicate, on the MPAT membrane in a 6 column and 4 row matrix, and assayed with the mAb of the present invention.

Right panel: MPAT immunodetection of the secreted Marker in the tissue culture supernatants of 12 cancer cell lines using mAb BF819 of the present invention, as described in Example 8. Spot intensity relates to expression levels.

FIG. 8: Immunodetection of secreted BF819 from cervical cancer cell lines

Left panel: Diagram illustrating the spot location of 4 cervical cancer cell lines in the MPAT assay. Equal amounts of proteins extracts of each cell line are spotted, in duplicate, on the MPAT membrane in a 6 column and 4 row matrix, and assayed with mAb BF819. Proteins are from tissue culture supernatants with (s+) or without (s−) fetal calf serum (FCS), or from cell extracts (x), as indicated. Cell lines are: Ca Ski (a), ME-180 (b), C-33A (c), and SiHa (d).

Right panel: MPAT immunodetection of the Marker either secreted in the tissue culture supernatants or expressed in cervical cancer cell lines, using mAb BF819, as described in Examples 8 and 5, respectively. Spot intensity relates to expression levels.

FIG. 9: Immunodetection of secreted BF819 in precipitated urine samples

Panel A: Diagram illustrating the spot location of 305 urine samples from a variety of cancer patients, benign and normal controls, comprising (Table 4): 35 colon cancer cases (including 12 stage I, 10 stage II, 12 stage III, 1 stage IV); 32 cases of inflammatory conditions of the colon (including 14 chronic colitis, 8 diverticulitis, and 10 Crohns disease); 10 cases of benign colon disease; 6 kidney cancers patients (including 3 stage I, 1 stage II, and 2 stage III); 1 kidney benign; 21 pancreatic cancer (mostly early stage, including 3 stage I, 15 stage II, 1 late and 2 unknown), and 1 benign pancreatic tumor; 107 prostate cancer patients (including 55 stage II, 45 stage III, and 7 stage IV) and 92 normal controls. Note that cancer stages I and II are defined as “early”, while stages III and IV are defined as “late”.

Panel B: urine samples were first acetone precipitated to concentrate proteins. Total proteins were measured and adjusted to 0.3 microgram per microliter. Then the same amount of precipitated protein from each of the 305 urine samples was spotted on the MPAT membrane in a double-blind experiment, incubated with mAb BF819 and detection was as described in Example 10.

FIG. 10: Detection of CEA in urine from cancer patients and normal controls

Panel A: Diagram illustrating the spot location of 305 urine samples, type and number as described in detail in FIG. 9 legend. Panel B: urine samples were first acetone precipitated to concentrate proteins; then the same protein amount was spotted on the MPAT membrane and assayed with a commercial mAb against CEA (ATCC), as described in Example 10.

FIG. 11: Detection of PSA in urine from cancer patients and normal controls

Panel A: Diagram illustrating the spot location of 305 urine samples, type and number as described in detail in FIG. 9 legend. Panel B: urine samples were first acetone precipitated to concentrate proteins; then the same protein amount was spotted on the MPAT membrane and assayed with a commercial mAb against PSA (ATCC), as described in Example 10.

FIG. 12: Immunodetection of secreted BF819 in unprecipitated urine samples

Panel A: Diagram illustrating the spot location of 47 unprecipitated urine samples (Table 4) from patients with: pancreatic cancer (n=5; 4 stage II and 1 unknown), colon cancer (n=10; all stages I or II), colon inflammatory diseases (n=5), colon benign conditions (n=4), prostate cancer (n=13; all stage II except D9 and D10 of stage III) and normal controls (10). Note that cancer stages I and II are defined as “early”, while stages III and IV are defined as “late”.

Panel B: urine samples were centrifuged to remove debris, yet not acetone precipitated, then spotted on the MPAT membrane in a double-blind experiment, in three different conditions: as is, or diluted 1:2 or 1:10 in Tris-Triton buffer as indicated. Protein samples were then incubated with mAb BF819 and detection was as described in Example 10.

### TABLE 1: CLINICAL SAMPLES FROM PATIENT TISSUES

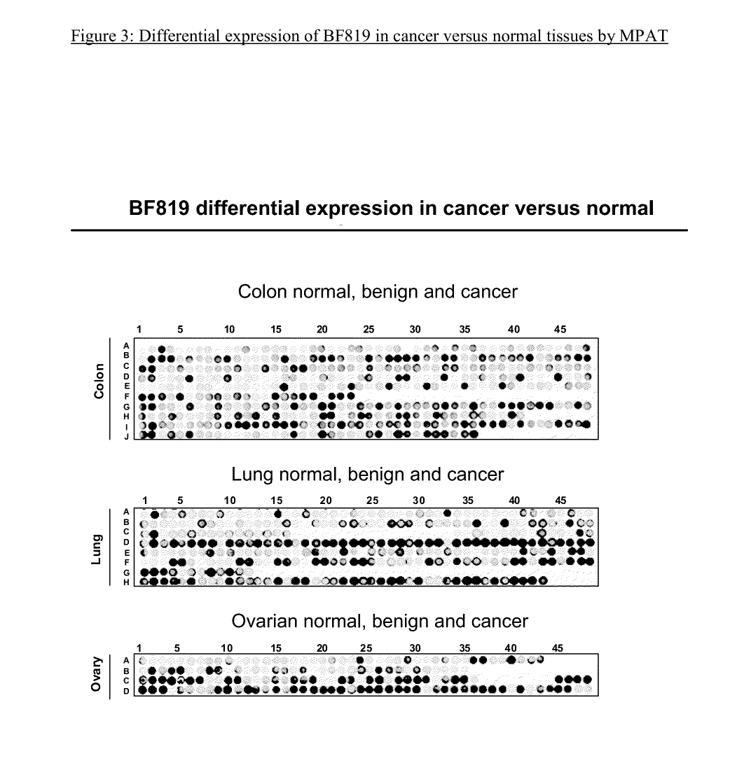

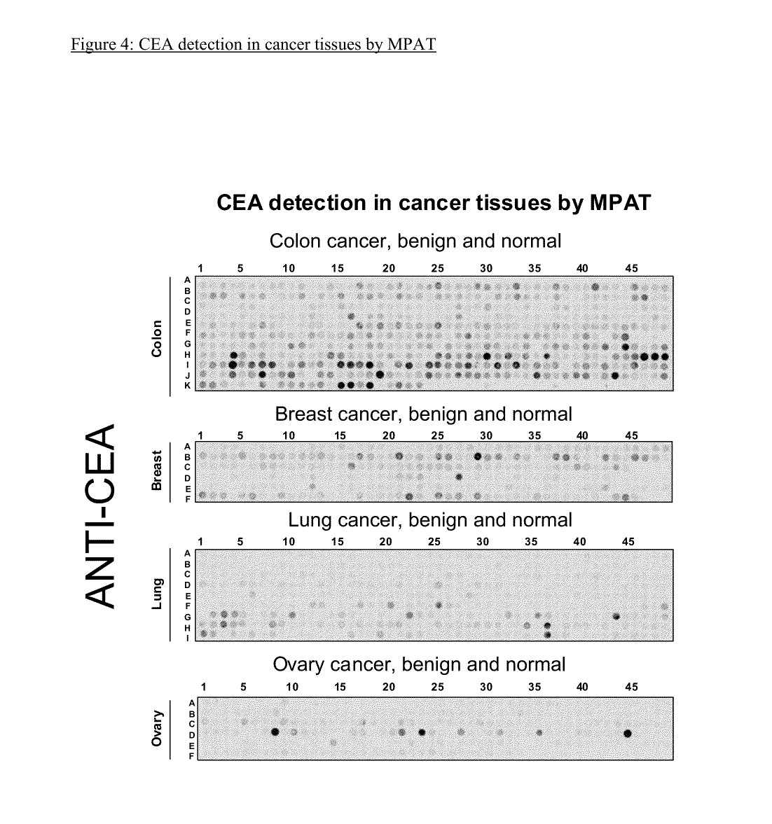

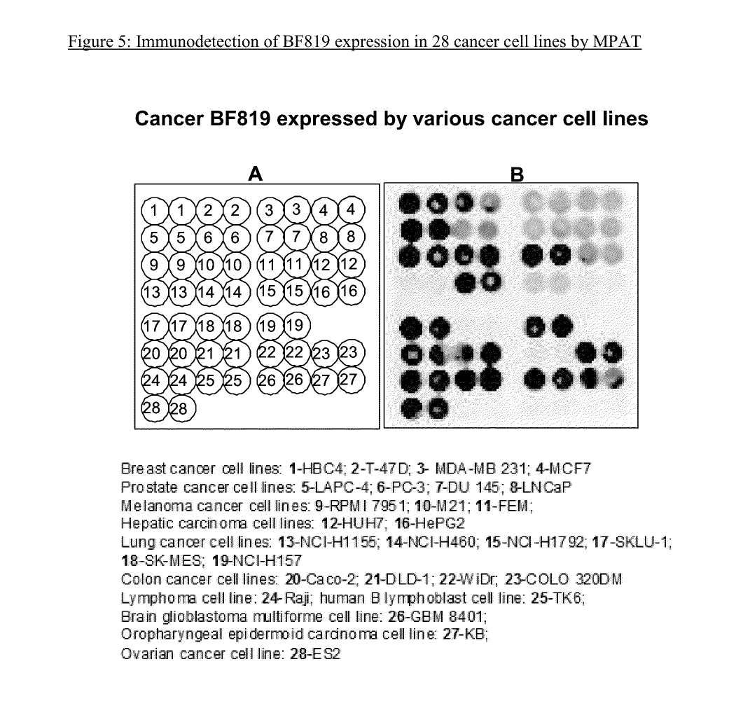

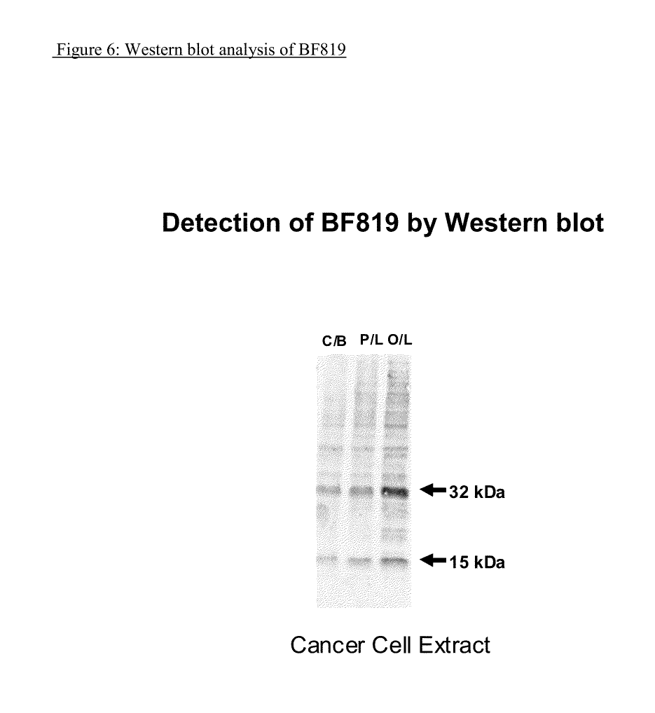

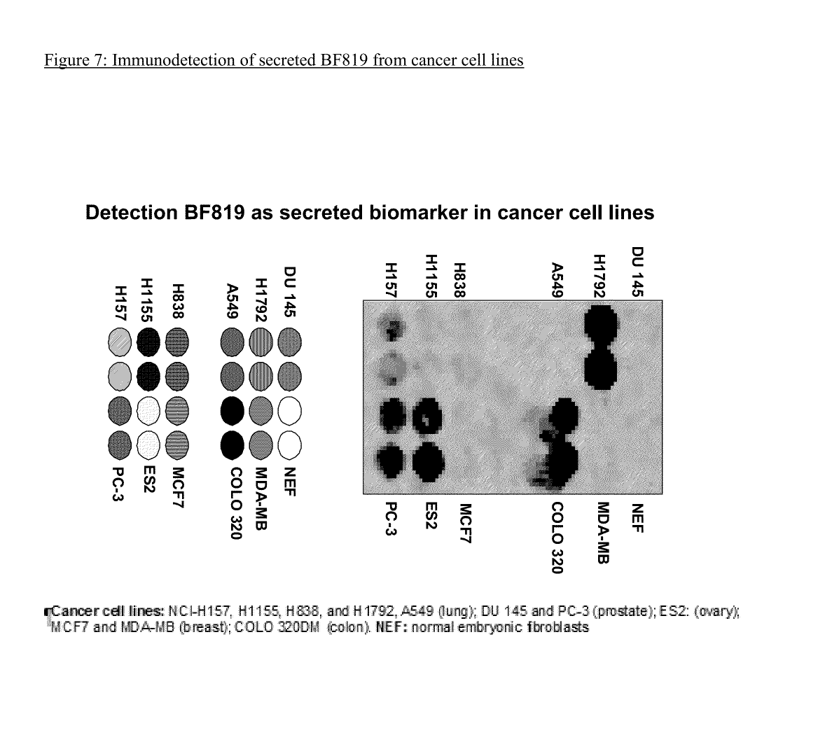

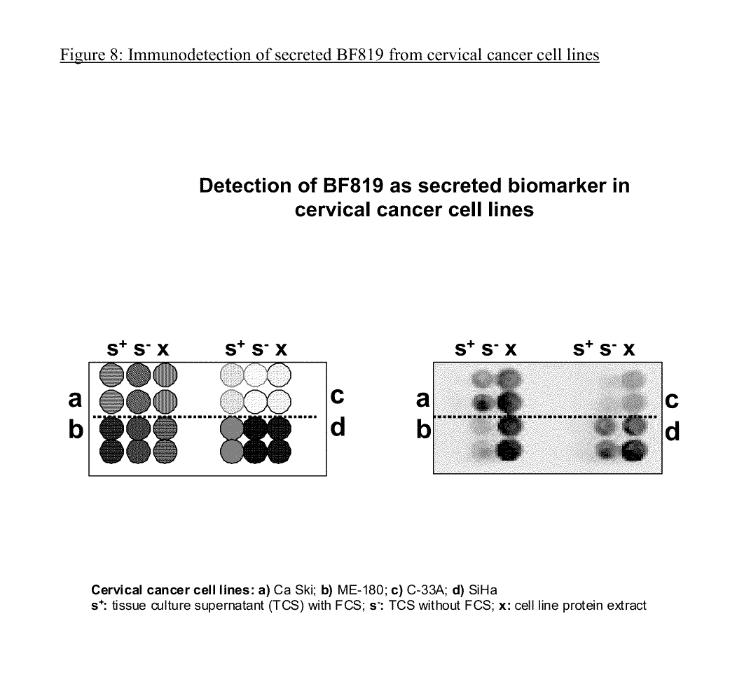

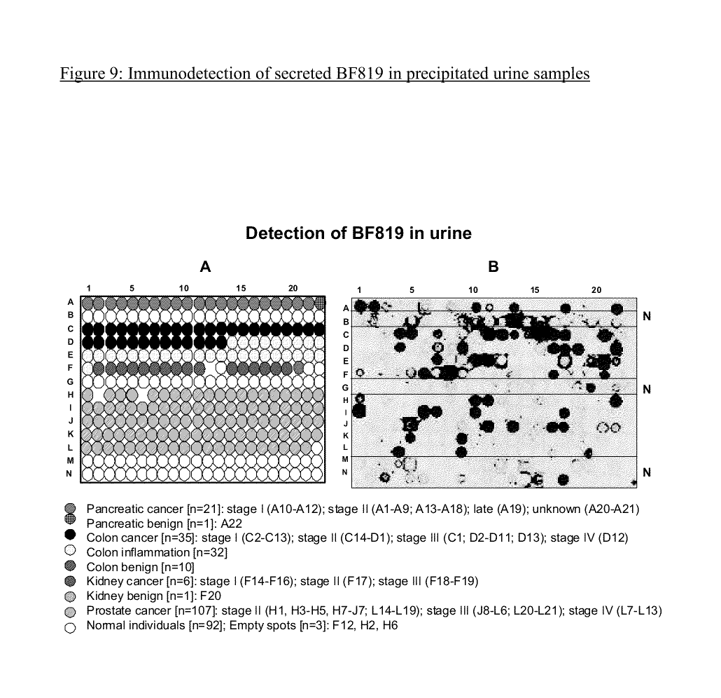

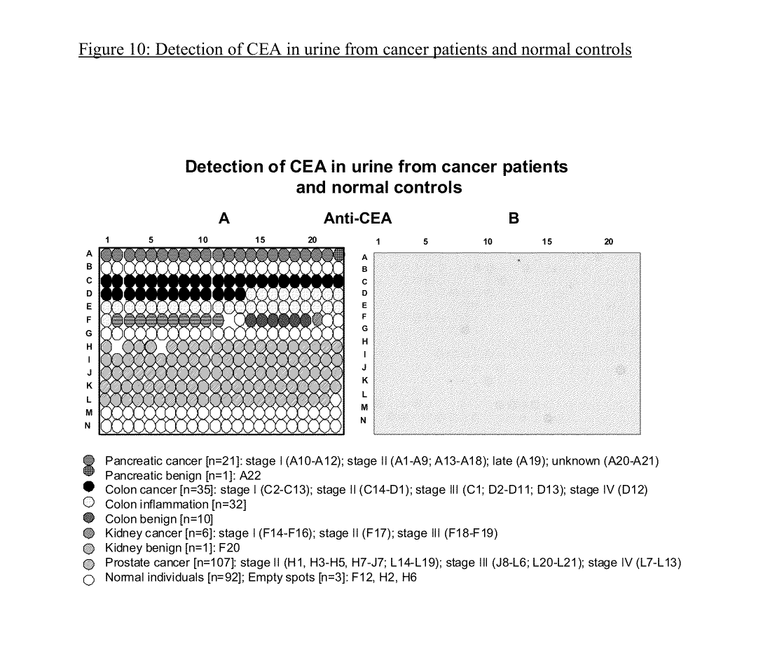

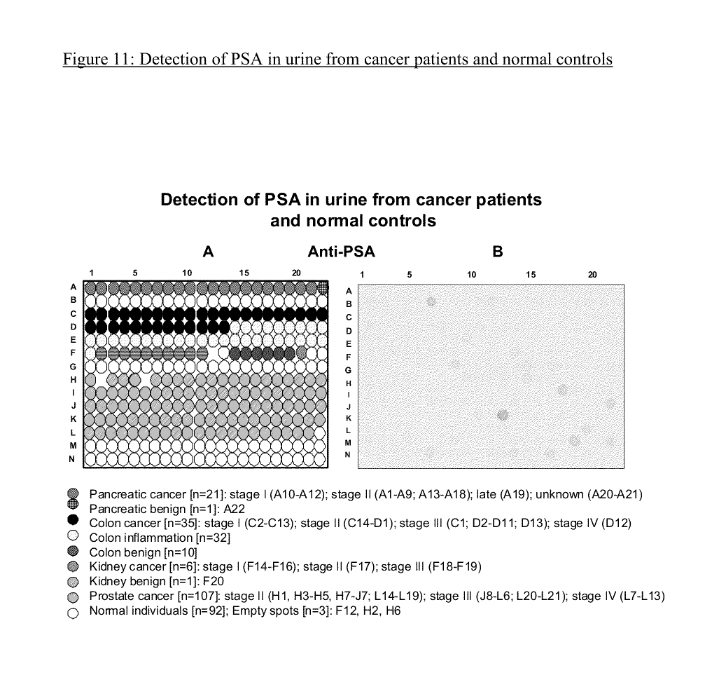

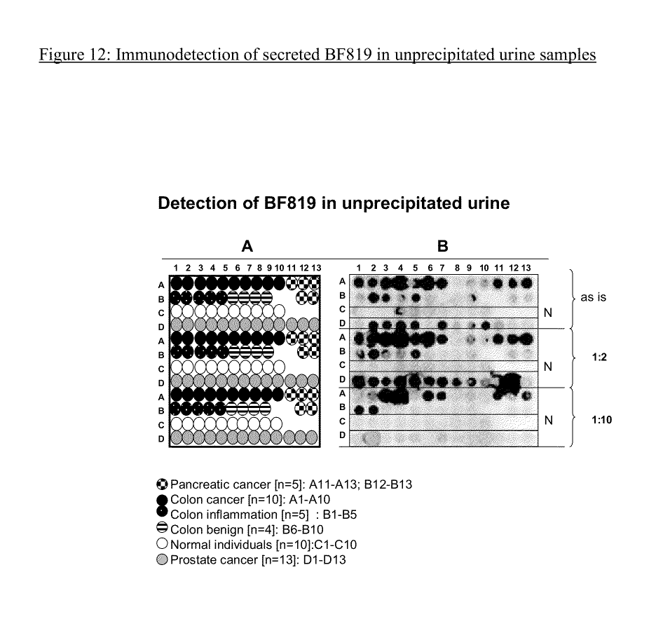

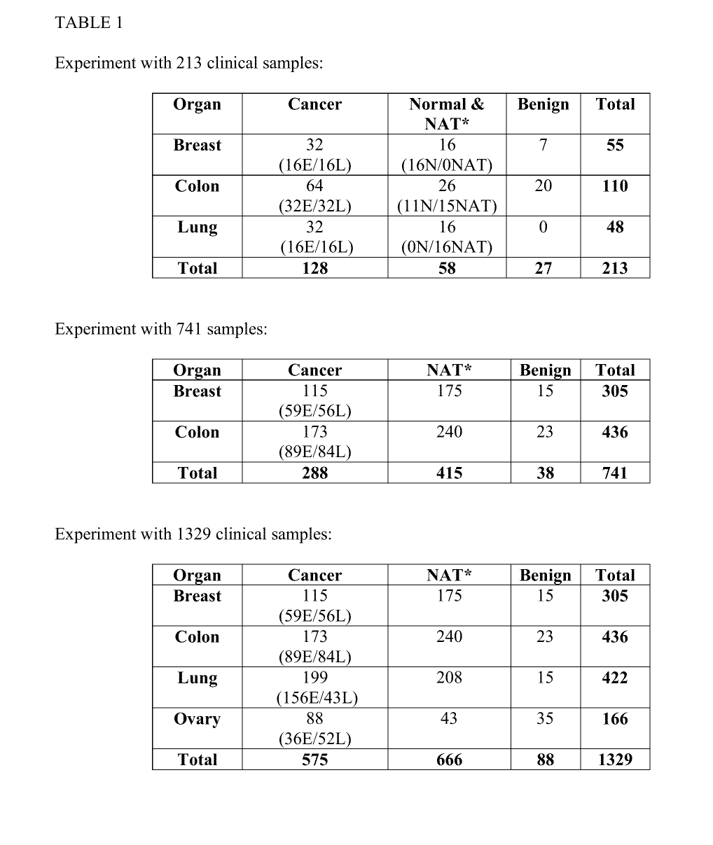

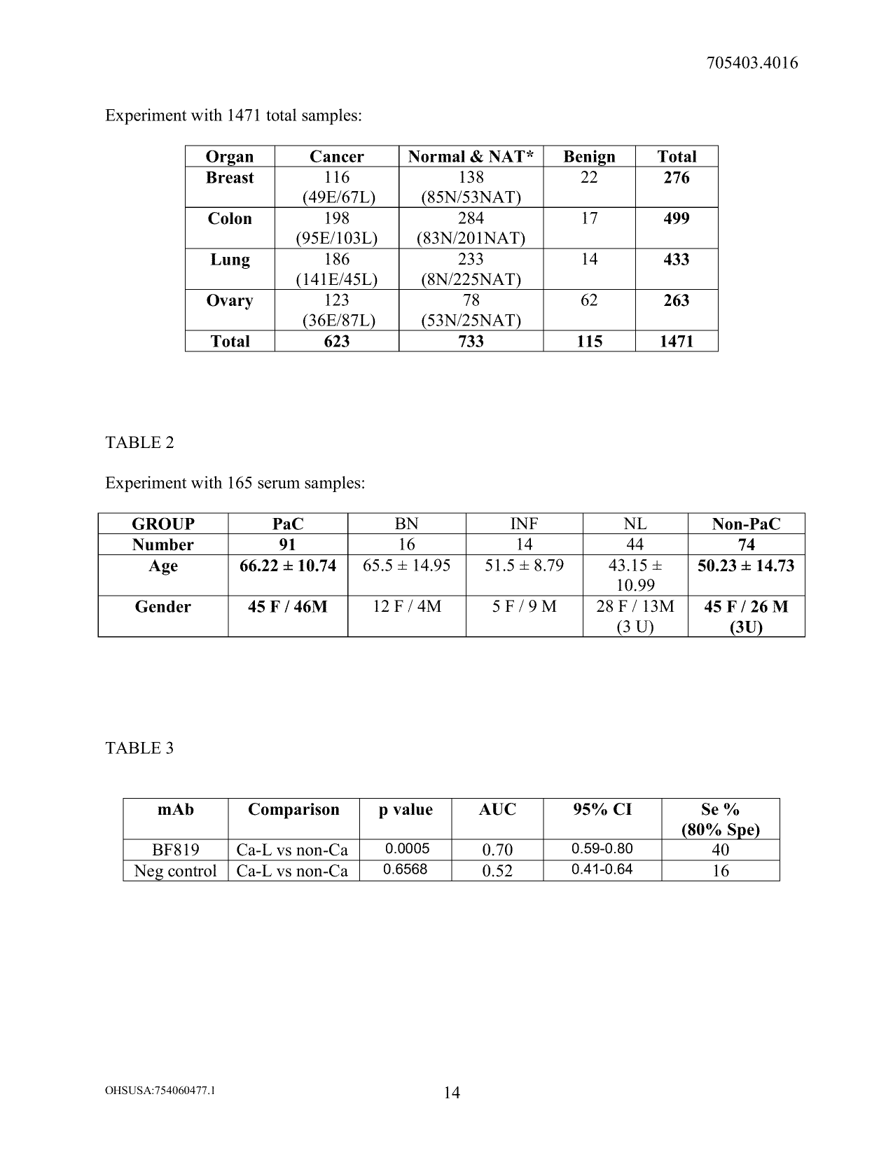

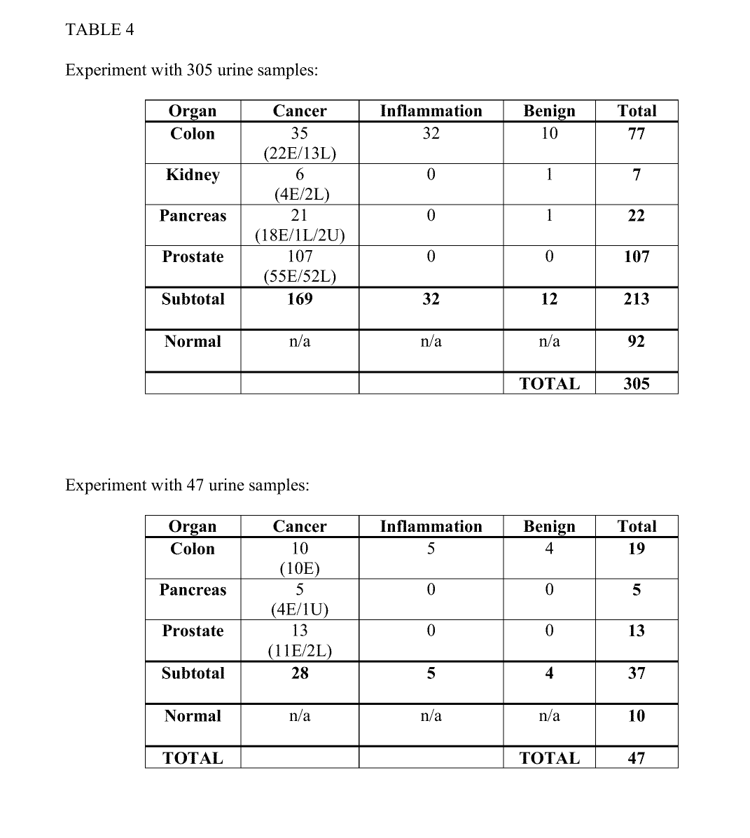

The following tables summarize the composition of the clinical sample sets used to determine differential expression of BF819 in cancer versus normal tissues by MPAT, as described in Example 4 and illustrated in FIG. 3. Clinical samples were frozen tissue biopsies of normal, benign and cancer patients provided with extensive clinical information and annotated pathology report, and protein extracts were prepared according to Example 4. Four different sets of clinical samples were used to confirm the diagnostic clinical utility of BF819. For each relevant organ site, the total number of specimens, the number of normal, benign and cancer samples, including early and late stage patients is indicated. N: normal tissues; NAT*: “normal adjacent to tumor”, i.e. normal tissue deriving from the same patient from which the tumor tissue is derived; E: early stage cancer; L: late stage cancer.

### TABLE 2: CLINICAL SAMPLES FROM PATIENT SERA

This Table summarizes the composition and clinical information of the 165 serum sample set used to determine differential expression of BF819 in patients with pancreatic cancer versus normal controls using the MPAT as described in Example 9. Information includes number, gender (F: female; M: male; U: unknown), and average±standard deviation of age (years) in the pancreatic cancer (PaC), and in the non-PaC group, including benign (BN), inflammation (INF) and normal (NL) controls. The PaC group includes 50 early stage, 34 late stage, and 7 with no stage information; the INF group exclusively consists of pancreatitis cases, and the BN group of benign tumors of the pancreas.

### TABLE 3: BF819 PERFORMANCE IN SERUM

This table summarizes statistical data analysis related to the expression of BF819 in pancreatic cancer (Ca) and non-pancreatic cancer (non-Ca) patient serum as revealed by the MPAT experiment described in Example 9. Data provided are: p value, area under curve (AUC) and its 95% confidence interval (CI), and sensitivity value at 80% specificity for the Bm of the present invention, and for the negative control (Neg control), in the late stage pancreatic cancer (Ca-L) versus non-pancreatic cancer control group (non-Ca) comparison.

### TABLE 4: URINE CLINICAL SPECIMENS

Table 4 summarizes the composition of the clinical sample sets used to detect the presence of BF819 in urine of cancer patients, benign and normal controls, as illustrated in FIGS. 9 to 12. Two sets of urine specimens were used, comprising either 305 or 47 samples; they were either acetone-precipitated to concentrate proteins, or unprecipitated, respectively, as described in Example 10. Details on disease stage and inflammatory conditions are provided in FIGS. 9 to 12 and their legends. E: early stage cancer (stages I and II); L: late stage cancer (stages III and IV); U: unknown.

### DETAILED DESCRIPTION OF THE INVENTION

### BF819

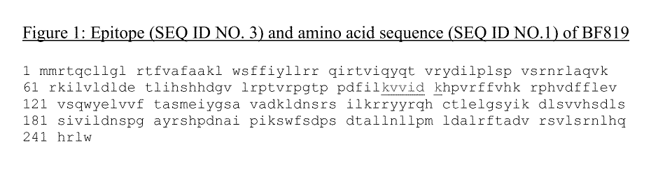

The present invention relates to composition and methods using a biomarker, designated BF819 (SEQ ID NO: 1) for early detection of cancer including, antibodies against the marker, and specifically a novel monoclonal antibody (mAb) that recognizes a specific epitope on BF819 (SEQ ID NO: 3). The amino acid sequence (SEQ ID NO: 3) of the epitope on BF819 recognized by the novel mAb of the present invention is underlined in FIG. 1. This marker is differentially expressed (over-expressed) in individuals with cancer as compared to individuals without cancer (individuals without cancer are interchangeably referred to herein as “normal”, “control”, or “healthy” individuals).

BF819 may be used in a variety of clinical indications for cancer, including, but not limited to, detection of cancer (such as in an asymptomatic individual or population or in a high-risk individual or population), characterizing cancer (e.g., determining cancer type, sub-type, or stage) such as distinguishing between non-small cell lung cancer (NSCLC) and small cell lung cancer (SCLC) and/or between adenocarcinoma and squamous cell carcinoma (or otherwise facilitating histopathology), determining whether a lesion is a benign lesion or a malignant tumor (including using mAbs to BF819 for imaging), cancer prognosis, monitoring cancer progression or remission, monitoring for cancer recurrence, monitoring metastasis, treatment selection, monitoring response to a therapeutic agent or other treatment, stratification of patients for MRI or computed tomography (CT) screening (e.g., identifying those patients at greater risk of cancer and thereby most likely to benefit from enhanced screening, thus increasing the positive predictive value of any parallel screening method), combining BF819 testing with supplemental biomedical parameters such as toxin exposure, smoking history, BRCA-1 or -2 presence, PSA scores or any of the existing markers noted below, or with tumor or nodule size, tumor morphology, etc. (such as to provide an assay with increased diagnostic performance compared to another testing technique alone or in combination with BF819), facilitating the diagnosis of a biological sample as malignant or benign, facilitating clinical decision making once a cancer is observed by margins, or of biopsy if the sample is deemed medium to high risk, and facilitating decisions regarding clinical follow-up (e.g., whether to implement repeat detection of this or another marker, imaging, biopsy, or other measure).

BF819 may be quantified when diagnosing cancer such that a high or low abundance level in an individual who is not known to have cancer may indicate that a threshold amount present in a sample from the individual correlates to cancer at a specific stage, thereby enabling early detection of cancer at an early stage of the disease when treatment is most effective, i.e. perhaps before the cancer is detectable by other techniques or before other symptoms appear. An increase in the abundance of BF819 may be indicative of cancer progression, e.g., a tumor or abnormal tissue is growing and/or metastasizing (and thus a poor prognosis), whereas a decrease in the abundance of BF819 may be indicative of cancer remission, e.g., a tumor is shrinking (and thus a good prognosis). Similarly, an increase in the abundance of BF819 during the course of cancer treatment may indicate that the cancer is progressing and therefore indicate that the treatment is ineffective, whereas a decrease in the abundance of BF819 during the course of cancer treatment may be indicative of cancer remission and therefore indicate that the treatment is working successfully. Additionally, an increase or decrease in the abundance of BF819 after an individual has apparently been cured of cancer may be indicative of cancer recurrence or metastasis. Detection of “differential” expression, or variation from a “normal” expression level, can also be used for another purpose described herein.

Detection of BF819 may be particularly useful following, or in conjunction with cancer treatment, such as to evaluate the success of the treatment or to monitor cancer remission, recurrence, and/or progression (including metastasis) following treatment. Cancer treatment may include, for example, administration of a therapeutic agent to a patient, surgery (e.g., surgical resection of at least a portion of abnormal tissue or a tumor), radiation therapy, or any other type of cancer treatment used in the art, and any combination of these treatments.

Antibodies to BF819 may also be used in imaging tests. For example, an imaging agent can be coupled to anti-BF819 mAbs, which can be used to aid in cancer screening or diagnosis, to monitor disease recurrence, progression/remission or metastasis, to plan surgery, biopsy, or radiation therapy, or to monitor response to therapy, among other uses. The mAbs disclosed herein are formulated to enhance stability, reduce immunogenicity and enhance plasma half-life, pH-range stability and other desirable pharmacological parameters by techniques known in the art.

As used herein the term “antibody” refers to a polyclonal, monoclonal, recombinant antibody, full-size molecule or antibody fragment thereof, including but not limited to Fab′″, scFv, single chain variable fragment, affibodies, diabodies, or any other antibody fragment, or any other recombinant version of conventional or combinatorial antibody, as well as any single or double chained binding agents comprised of a variety of known structures, including another molecule or biologically compatible tag that facilitates detection of the antibody while retaining the ability of the antibody to recognize the relevant epitope or BF819 to a sufficient extent for detection to occur.

Unless specified, the term “antibody” is used interchangeably herein to refer to any of the above species. The novel compositions are comprised of non-naturally occurring species of a mAb capable of binding BF819. Methods of the invention include use of both naturally occurring and synthetic variants of mAb BF819. Thus, “antibodies” include antibodies produced in vitro, as well as antibodies generated in vivo by injection of BF819 or a polynucleotide encoding BF819 in a mammal capable of mounting a sufficient immune response to yield high titre IgG antibodies. Methods to produce polyclonal, monoclonal, recombinant antibodies and fragment thereof are know to the skilled in the art (Coligan et al, Current Protocols in Immunology, Wiley Intersciences; Kohler et al. Nature 256:495-497, 1975; Phage display of peptides and proteins—A laboratory manual, Kay B. B., Winter J. & McCafferty J., Eds, Academic Press, 1996).

The term “monoclonal antibody”, as used herein, refers to a novel antibody obtained from a population of substantially homogeneous antibodies, i.e., the individual antibodies comprising the population are substantially identical except for naturally occurring mutations present in minor amounts. Monoclonal antibodies are highly specific and are typically directed against a single epitope and variants thereof as described below. Furthermore, in contrast to polyclonal antibody preparations, which typically include different antibodies directed against different determinants (epitopes), each monoclonal antibody is directed against BF819 (SEQ ID NO: 1) and ideally specified for the defined epitope (SEQ ID NO: 3) or variants thereof as described herein. In addition to specificity, the monoclonal antibody against BF819 described herein is substantially homogenous and is produced by an available hybridoma. The modifier “monoclonal” indicates that the anti-BF819 antibody exists in a substantially homogeneous population of antibodies, but is not to be construed as requiring production of the antibody by any particular method.

An “isolated” or “purified” antibody is one that has been identified and separated and/or recovered from a component of the environment in which it is produced. Contaminant components of its production environment are materials that would interfere with diagnostic or therapeutic uses for the antibody, and may include enzymes, hormones, and other proteinaceous or nonproteinaceous solutes. In exemplary embodiments, the antibody can be purified as measurable by any of at least three different methods: 1) to greater than 95% by weight of antibody as determined by the Lowry method, preferably more than 99% by weight; 2) to a degree sufficient to obtain at least 15 residues of N-terminal or internal amino acid sequence by use of a spinning cup sequenator or 3) to homogeneity by SDS-PAGE under reducing or non-reducing conditions using Coomassie blue or silver stain. Isolated antibody can include an antibody in situ within recombinant cells since at least one component of the antibody's natural environment will not be present. Ordinarily, however, an isolated antibody can be prepared by at least one purification step.

A “transformed antibody” is a binding protein produced in a (host) species other than the species of the antigen (target) to which the antibody specifically binds. The transformed antibody typically has chemical or structural signatures characteristic of the host that do not exist in the target species. An example is a “transformed” BF819 mAb to the human BF819 protein produced in a bacterial species such as an E Coli or in a mammalian species such a CHO cell having glycosylation or other chemically distinct signatures compared to an anti-BF819 antibody existing in a mammal or vertebrate.

“Antibody specificity” refers to an antibody that has a stronger binding affinity for BF819 antigen from a first individual species than it has for a homologue of BF819 from a second species. Typically, an anti-BF819 antibody “binds specifically” to a human BF819 antigen (e.g., has a binding affinity (Kd) value of no more than about 1×10-⁷ M, preferably no more than about 1×10⁻⁸ M, and most preferably no more than about 1×10-⁹ M) but has a binding affinity for a homologue of the antigen from a second individual species at least about 50-fold, or at least about 500-fold, or at least about 1000-fold, weaker than its binding affinity for the human BF819.

An antibody “selectively” or “specifically” binds the BF819 a marker protein when the antibody binds the marker protein and does not significantly bind to unrelated proteins. An antibody can still be considered to selectively or specifically bind a marker protein even if it also binds to other proteins that are not substantially-homologous with the marker protein as long as such proteins share substantial homology with a fragment or domain of the marker protein epitope. Antibody binding to the marker protein is still selective and “specific” despite some degree of cross-reactivity to other antigens.

The term “epitope” is used to refer to the amino acid sequence within the marker polypeptide recognized by the mAb disclosed herein. The term “epitope”, “antigenic determinant”, “structural domain”, “antibody target” are interchangeably used to indicate the amino acid sequence, whether in isolated form or embedded in a polypeptide sequence or fragment and derivative thereof, which is recognized by the mAb. Epitopic determinants can be active surface groupings of molecules such as amino acids or sugar side chains and may have specific three-dimensional structural characteristics or charge characteristics.

The epitopes encompassed by the present invention comprise the epitope sequence (SEQ ID NO: 3) for BF819 (SEQ ID NO: 1), as well as any other sequence with 70%, 80%, 85%, 90%, 91%, 92%, 93%, 94%, 95%, 96%, 97%, 98% or 99% homology thereto and all inclusive values therein including translations and extensions at either end of the defined consensus epitope sequence, said homology including amino acid changes, preferably involving conservative amino acid substitutions, but including any amino acid substitution that maintains binding functionality, such as permutations, deletions, or insertions. As a result, alterations to the sequence of the epitope may exist as long as the mAb BF819 retains binding specificity as determined by the ability of the mAb to bind the BF819 marker at the altered epitope to form a complex in such a way that the binding event is detectable.

The terms “natural polynucleotide”, or “natural nucleotide sequence”, are used interchangeably herein and may include naturally occurring DNA sequences or downstream transcripts such as pre-RNA. The “natural polynucleotide” described herein is DNA, including genomic DNA, double or single-stranded, whether coding or non-coding strands, or RNA, including heteronuclear RNA, messenger RNA (mRNA), or any other form of RNA, such as small, anti-sense, interfering or silencing RNA whose espression correlates to the presence or espression of BF819 in vivo or in a biological sample.

A “synthetic polynucleotide” or “polynucleotide construct” may contain introns, 5′ and 3′ non-coding sequences, 5′ and 3′ transcriptional regulatory sequences, such as promoters, enhancers, polyadenylation signals, or translational control elements not present in the natural polynucleotide encoding the polypeptide marker as expressed in a human patient. The synthetic polynucleotide may include “natural polypeptide” sequences for BF819 that are manufactured to include DNA constructs to facilitate expression or regulation or that encode for leader or secretory sequences at the level of the polypeptide, or for an active or inactive pro-protein that is later processed into active or inactive shorter polypeptides. The assembled synthetic construct is constructed and oriented to facilitate expression of the natural polypeptide in a non-natural environment.

The synthetic polynucleotide described herein includes engineered splice variants and non-naturally-occurring allelic variants, and any non-natural variants encoding the biomarker of the present invention with a different nucleotide sequence due to the degeneracy of the genetic code. Variants encode fragments, analogs and derivatives of the marker, and may include deletion, substitution, addition or insertion variants created by design even if duplicated by unusual and rare phenomena including those created. The synthetic polynucleotide encompassed by the claims includes any length of said polynucleotide sequence, whether 5′ terminal, 3′ terminal or internal and transformed into entities chemically suited for use in a diagnostic platform.

Synthetic polynucleotides of the present invention, including DNA constructs can be manufactured using standard molecular biology techniques and the sequence information described herein (Sambrook et al., 1989, Molecular Cloning: A Laboratory Manual, 2^{nd} ed., Cold Spring Harbor Laboratory Press, Cold Spring Harbor, N.Y.).

The protein marker BF819 is substantially free of cellular material or free of chemical precursors or other chemicals. BF819 proteins can be purified to homogeneity or other degrees of purity. The level of purification can be based on the intended use. The primary consideration is that the preparation allows for the desired function of the protein, even if in the presence of considerable amounts of other components.

To determine the percent identity of two amino acid sequences i.e. a reference and a test sequence such as the naturally occurring BF819 polynucleotide and another sequence such as a synthetic sequence, the sequences can be aligned for optimal comparison purposes (e.g., gaps can be introduced in one or both of a first and a second amino acid or polynucleotide sequence for optimal alignment and non-homologous sequences can be disregarded for comparison purposes). In an exemplary embodiment, at least 30%, 40%, 50%, 60%, 70%; 80% or 90%, 91%, 92%, 93%, 94%, 95%, 96%, 97%, 98%, 99% or 100% of the length of a reference sequence can be aligned for comparison purposes. A test sequence may also be tested for equivalent reactivity, including specific reaction of a test polypeptide or polypeptide encoded by a test polynucleotide, with an antibody to BF819, particularly a novel mAb binding at the epitope described herein. The amino acid residues or nucleotides at corresponding amino acid positions or nucleotide positions are compared and relative functionality analyzed by techniques known in the art. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “homology”). The percent identity between the two sequences is a function of the number of identical positions shared by the sequences, taking into account the number of gaps, and the length of each gap, that are introduced for optimal alignment of the two sequences.

The monoclonal antibody disclosed herein is mAb BF819 and is preferably identified by its ability to bind to the epitope (SEQ ID NO: 3) it recognizes (FIG. 1). The antibodies encompassed by the present invention include all antibodies, as defined above, that are capable of binding (having specific binding affinity) to BF819, both naturally occurring in humans and synthesized by known chemical or biological techniques, and preferably those polypeptide variants containing the epitope (SEQ ID NO: 3).

The reactivity of mAb BF819 is specifically targeted to the BF819 polypeptide (SEQ ID NO: 1) encoded by the polynucleotide (SEQ ID NO: 2) encoding the BF819 marker, as long as the BF819 species harbors an epitope, facilitating use of the marker in any embodiment of the present invention. The specific novel mAb disclosed herein and exhibiting binding affinity to the epitope (FIG. 1, SEQ ID NO: 3) may also be reactive against proteins or fragments thereof that share substantial similarity in antigenic determinants or structural domains (substantially similar epitopes.) Indeed it is well-established and known to those skilled in the art that protein families performing similar cellular functions share functional domains in the form of highly conserved amino acid sequence motifs, which become the signature of that given protein and their variants function (polymerase, kinase, protease, etc.). Hence, related target polypeptides may share amino acid motifs or functional domains with BF819.

A “biological sample(s)” as referred to herein is a quantity of tissue, or body fluid or other material from human patient or normal controls, and comprises tissues and/or biological fluids containing a polypeptide expressed by the patient. Tissue samples include, but are not limited to fresh or frozen normal or diseased tissues (including normal, tumor adjacent tissues), particularly cancer tissues, such as derived from a tumor biopsy cell line (lysate or intact) extracts, including the extracts of the MPAT assay described below, or any other preparation that may be processed for advantageous use in the methods or kits of the invention, and including from different organ sites, different histological types of cancer, and different stages (early, advanced, metastatic), but also tissues from benign and/or inflammatory conditions at a given organ site. A “patient test sample(s)” includes any body fluid, obtained by a non-invasive sampling method, used for detection of secreted proteins, including but not limited to, urine (precipitated or unprecipitated), plasma, serum, blood, saliva, sputum, nipple aspirate fluid, any lavages (such as but not restricted to ductal lavages) or bronchio-alveolar lavages. The term “patient” refers to a human previously diagnosed with disease or an asymptomatic person screened for disease.

In preferred embodiments, the biological samples examined are matched normal and tumor tissues derived from the same patient including, normal adjacent tumor, samples derived from the same or different cancer patients. Samples may include primary tumor or metastasis, early or late stages of cancer, from stage I to stage 1V, as well as benign tumors and inflammatory conditions. For the purpose of the present invention, biological samples referred herein may also include mammalian cell cultures, preferably cancer cell lines, as well as microdissected cell types from normal or disease tissue samples, or from a given subcellular compartment.

BF819 Polypeptide.

The present invention includes compositions comprising and methods using a polypeptide having the amino acid sequence (SEQ ID NO: 1) and whose expression levels are altered in human cancer. The consensus amino acid sequence (SEQ ID NO: 1) recognized by mAb BF819 results from the phage display approach of Example 1 and is the “consensus epitope” for the mAb of the present invention. The “consensus epitope” sequence (SEQ ID NO: 3) is present in the BF819 marker. The protein identity of BF819 is validated by BLAST search, in the NCBI protein database, using the optimal consensus epitope sequence (SEQ ID NO: 3) as described in detail in Example 2. Specifically, the consensus epitope or, alternatively, the 12-mer peptide with the best ELISA results, is entered in a BLAST search (blast.ncbi.nlm.nih.gov) to retrieve all possible proteins with highest homology to the consensus epitope (SEQ ID NO: 3) or queried peptide. Marker identity as described herein and in the cited references and its protein sequence is determined upon correlation with other protein data (western blot, molecular weight, subcellular localization, biomarker expression by IHC etc.) and other databases, such as Human Protein Atlas and UniProt database.

BF819 is CTD nuclear envelope phosphatase 1 (CTDNEP1), also known as serine/threonine-protein phosphatase dullard, composed of 244 amino acids, with expected MW of 28,377 Da (UniProt 095476; NP_{—}056158). Dullard forms an active phosphatase complex with CNEP1R1 to dephosphorylate and activate lipin (RefSeq; Pruitt, 2012). Dullard participates in a unique phosphatase cascade regulating nuclear membrane biogenesis, a cascade that is conserved from yeast to mammal (Kim, 2007). Recently, mutations in the CTDNEP1 gene have been linked to medulloblastoma (Jones, 2012). The amino acid sequence of BF819 is provided in FIG. 1. (SEQ ID NO: 1).

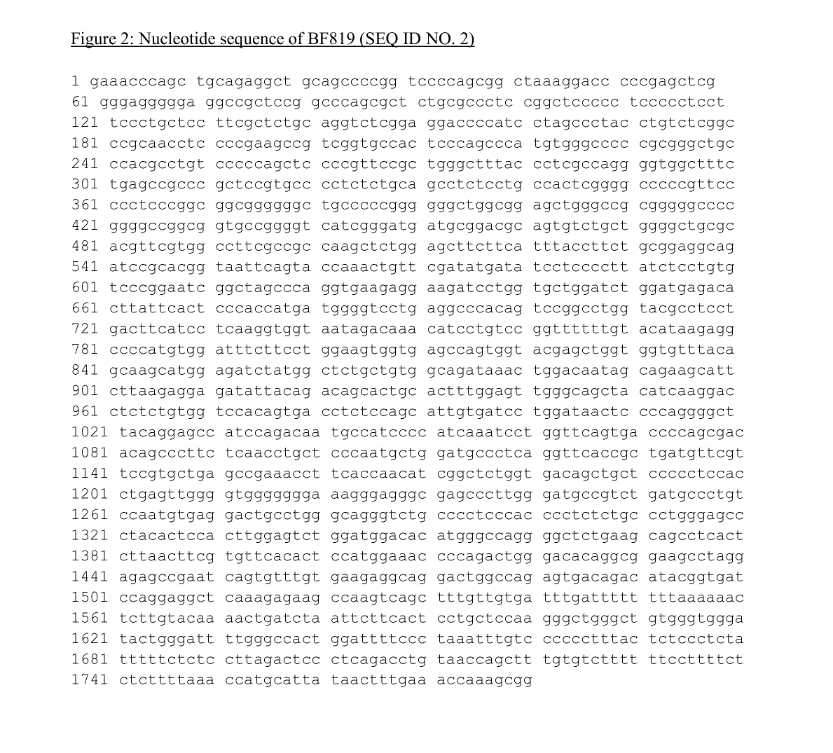

In part, the present invention comprises the BF819 amino acid sequence (SEQ ID NO: 1) as well as a population of polypeptides having related or identical polypeptide sequences that can be encoded by the BF819 polynucleotide sequence (SEQ ID NO: 2) identified in FIG. 2 such as isoforms, fragments, variants and derivatives thereof, and related polypeptide variants having a defined epitope (SEQ ID NO: 3).

The composition of BF819 polypeptides includes any non-naturally occurring species manufactured from the synthetic polynucleotide sequences claimed herein by conventional and non-conventional mechanisms, such as frameshift, either occasional or programmed, internal initiation, or non Watson-Crick codon-anticodon pairing events at the translation level. These and other mechanisms may lead to the production of hybrid or synthetic polypeptides of BF819, for example carrying amino acid motifs of one reading frame and/or amino acid motifs expressed from another reading frame. Such hybrid proteins carrying multiple amino acid domains may consequently be regulated according to as many different functional domains as featured in the hybrid polypeptide. Synthetic or hybrid polypeptides may retain substantially the same biological function or activity as the relevant biomarker while partially differing in any degree from the natural amino acid sequence.

Non-naturally occurring variants of the BF819 protein can readily be generated using recombinant techniques. Such variants include, but are not limited to, deletions, additions, and substitutions in the amino acid sequence of the BF819 protein. For example, one class of substitutions is conserved amino acid substitutions. Such substitutions are those that substitute a given amino acid in BF819 by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent are found in Bowie et al., Science 247:1306-1310 (1990).

Amino acids that are essential for function can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity or in assays such as in vitro proliferative activity. Sites that are critical for binding partner/substrate binding can also be determined by structural analysis such as crystallization, nuclear magnetic resonance, or photoaffinity labeling (Smith et al., J. Mat. Biol. 224:899-904 (1992); de Vos et al., Science 255:306-312 (1992)).

Compositions, variants or fragments of naturally occurring BF819 useful in the methods of the invention typically comprise at least about 5, 6, 8, 10, 12, 14, 16, 18, or more contiguous amino acid residues of BF819. Such fragments can be chosen based on the ability to retain one or more of the biological activities of BF819 or can be chosen for the ability to perform a function, e.g., bind a substrate or act as an immunogen. Particularly important fragments are biologically active fragments, such as peptides that are, for example, about 8 or more amino acids in length. Such fragments can include a domain or motif of BF819, e.g., an active site, a transmembrane domain, or a binding domain. Further, possible fragments include, but are not limited to, soluble peptide fragments and fragments containing immunogenic structures. Domains and functional sites can readily be identified, for example, by computer programs well known and readily available to those of skill in the art (e.g., PROSITE analysis).

Variants of the BF819 polypeptide may also be comprised of non-naturally occurring modifications to the BF819 polypeptide including, but not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, sialylation gamma carboxylation, glycosylation, GPI anchor formation, hydroxylation, iodination, methylation, myristoylation, oxidation, proteolytic processing, phosphorylation, prenylation, racemization, selenoylation, sulfation, tRNA-mediated addition of amino acids to proteins such as arginylation, and ubiquitination.

Such modifications are well known to those of skill in the art and have been described in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins-Structure and Molecular Properties, 2nd Ed., T. E. Creighton, W. H. Freeman and Company, New York (1993). Many detailed reviews are available on this individual, such as by Wold (Posttranslational Covalent Modification of Proteins, H. C. Johnson, Ed., Academic Press, New York 1-12 (1983)); Seifter et al. (Meth. Enzymol. 182: 626-646 (1990)); and Rattan et al. (Aim. N.Y. Acad. Sci. 663:48-62 (1992)).

BF819 polypeptides encompassed by the present invention may include fusion to a marker sequence supplied by an expression vector and enabling purification of the polypeptide of the present invention, such as hexa-histidine tag, glutathione-S-transferase, hematglutinin, luciferase, beta-galactosidase, and the like. The polypeptides may also include polypeptides, in full or in part, modified by any form of post-translational modification, such as phosphorylation, acylation, methylation, ubiquitination, etc., conjugation or covalent linkage to lipids, polysaccharides and the like. These polypeptides further include full-length mature folded proteins, or fragments thereof, either derived by internal initiation, early termination, degradation, or post-translational processing. Non-naturally occurring polypeptide variants of BF819 may be distinguished from naturally occurring forms by several parameters including characterizing unique sequence content, conjugation with other chemical species, alterations in glycosylation or other chemical signatures including sialylation, any altered structural or chemical composition resulting from expression in non-mammalian expression systems or organisms, altered folding characteristics from non-mammalian expression or processing including measured variances in folding structure caused by separation on a column or other purification or processing techniques.

The source of the polypeptides include a natural polypeptide purified from a biological mixture such as that of a protein extract from human specimens, or a recombinant polypeptide generated by various methods known in the art as described herein, or a purely synthetic polypeptide. Whether recombinant or synthetic, naturally-occurring BF819 polypeptides or BF819 variants can be generated based on the sequence information disclosed herein (SEQ ID NO: 1-N0:3 and FIGS. 1 and 2).

Variant polypeptides of BF819 also include isolated antigenic determinants, epitope sequences, or other structural protein domains, produced by different methods known those skilled in the art, including but not limited to: direct peptide synthesis using conventional solid-phase techniques (Merrifield, 1963), direct gene synthesis, in vitro run-off transcription from vectors carrying bacteriophage promoters, high-throughput cell-free translation systems (Sawasaki, 2002), and by recombinant techniques aiming at the expression and purification of recombinant proteins or protein fragments from bacterial, yeast, insect, or mammalian expression vectors that are commercially available and known to those in the art.

Variant polypeptides of BF819 can also be purified from cells that express it, purified from cells that have been altered to express it (recombinant), or synthesized using known protein synthesis methods (e.g., Sambrook et al., Molecular Cloning: A Laboratory Manual. 3rd. ed., Cold Spring Harbor Laboratory Press, Cold Spring Harbor, N.Y., (2001)). For example, a natural or synthetic polypeptide encoding the BF819 protein is integrated into an expression vector, the expression vector introduced into a host cell, and the non-naturally occurring BF819 polypeptide variants expressed in the host cell. The polypeptide variant can then be isolated from the cells by an appropriate purification scheme using standard protein purification techniques.

BF819 Polynucleotide.

One aspect of the present invention is a synthetic polynucleotide sequence (SEQ ID NO: 2) which encodes a gene product, namely BF819 (SEQ ID NO: 1) whose expression levels are altered in human cancer. The natural polynucleotide sequence of BF819 has been confirmed based on the amino acid sequence of the epitope (SEQ ID NO: 3) recognized by mAb BF819, as described in details in Examples 1 and 2. FIG. 2 provides the natural polynucleotide coding sequence (SEQ ID NO: 2) for Homo sapiens CTDNEP1 gene, encoding CTD nuclear envelope phosphatase 1 (Gene ID: 23399; NM_{—}015343.4).

BF819 Gene.

Exemplary BF819 nucleic acid molecules of the invention consist essentially of, or comprise a nucleotide sequence that encodes a BF819 protein of the invention, an allelic variant thereof, or an ortholog or paralog thereof for example. As used herein, a synthetic polynucleotide bears chemical signatures resulting from defined differences between the synthetic entity and the nucleic acid sequence of the natural polynucleotide. Preferably, the synthetic polynucleotide is free of sequences which naturally flank the nucleic acid (i.e. sequences located at the 5′ and 3′ ends of the nucleic acid) in the genomic DNA of the organism from which the natural polynucleotide is derived. The synthetic polynucleotide typically includes synthetic flanking sequences, particularly contiguous protein-encoding sequences and protein-encoding sequences within the same gene but separated by introns in the genomic sequence, and flanking nucleotide sequences that contain regulatory elements. The primary consideration is that the nucleic acid is distinguishable from the naturally occurring sequence by engineered or manufactured manipulations described herein including recombinant expression, the design and preparation of probes and primers, and other features such as a non-naturally occurring transcript/cDNA molecule, or synthetic polynucleotide produced by recombinant technique, or chemical synthesis.

A synthetic polynucleotide can be comprised of the naturally occurring polynucleotide and fused to other coding or regulatory sequences and still be considered synthetic. Synthetic polynucleotides can include heterologous nucleotide sequences, such as heterologous nucleotide sequences that are fused to a nucleic acid molecule by recombinant techniques. For example, recombinant DNA molecules contained in a vector are considered synthetic. Further examples of synthetic DNA molecules include recombinant DNA molecules maintained in heterologous host cells, or purified (partially or substantially) non-naturally-occurring DNA molecules in solution. Synthetic pre-RNA or RNA molecules include in vivo or in vitro RNA transcripts of synthetic DNA molecules as long as the species is not naturally occurring, but may include species produced by unusual or rare phenomenon. Synthetic nucleic acid molecules further include such variant molecules produced synthetically.

Synthetic polynucleotides encode a mature protein plus additional amino or carboxyl-terminal amino acids, or amino acids interior to the mature protein (when the mature form has more than one peptide chain, for instance). Such sequences may play a role in processing of a protein from precursor to a mature form, facilitate protein trafficking, prolong or shorten protein half-life, or facilitate manipulation of a protein for assay or production, among other things. As generally is the case in situ, additional amino acids may be processed away from the mature protein by cellular enzymes.

Synthetic nucleic acid molecules include, but are not limited to, sequences encoding a BF819 polypeptide variant alone, sequences encoding a mature protein with additional coding sequences (such as a leader or secretory sequence (e.g., a pre-pro or pro-protein sequence)), and sequences encoding a mature protein (with or without additional coding sequences) plus additional non-coding sequences (e.g., introns and non-coding 5′ and 3′ sequences such as transcribed but non-translated sequences that play a role in transcription, mRNA processing (including splicing and polyadenylation signals), ribosome binding, and/or stability of mRNA). In addition, synthetic polynucleotides can encode a BF819 polypeptide variant that facilitates purification.

Synthetic polynucleotides including cDNA and genomic DNA obtained by cloning or produced by chemical synthetic techniques or by a combination, can be double-stranded or single-stranded. Single-stranded nucleic acid can be the coding strand (sense strand) or the non-coding strand (anti-sense strand).

Synthetic polynucleotides are non-naturally occurring variants made by random or targeted mutagenesis techniques, including those applied to isolated nucleic acid molecules, cells, or organisms. Accordingly, nucleic acid molecule variants can contain nucleotide substitutions, and sequence deletions, inversions, and/or insertions can occur in either or both the coding and non-coding regions, and variations can produce conservative and/or non-conservative amino acid substitutions.

A fragment of a synthetic polynucleotide typically comprises a contiguous nucleotide sequence at least 8, 10, 12, 15, 16, 18, 20, 22, 25, 30, 40, 50, 100, 150, 200, 250, 500 (or any other number in-between) or more nucleotides in length and encodes epitope bearing regions of the encoded BF819 polypeptide particularly for separation of the protein from related isoforms or variants as DNA probes and primers.

A probe/primer typically comprises a substantially purified oligonucleotide or oligonucleotide pair. An oligonucleotide typically comprises a nucleotide sequence that hybridizes under stringent conditions to at least about 8, 10, 12, 14, 15, 16, 17, 18, 19, 20, 21, 22, 23, 24, 25, 30, 40, 50 (or any other number in-between) or more contiguous nucleotides.

As used herein, the term “hybridizes under stringent conditions” is intended to describe conditions for hybridization and washing under which nucleotide sequences encoding a protein at least 60-70% homologous to each other typically remain hybridized to each other. The conditions can be such that sequences at least about 60%, at least about 70%, or at least about 80% or more homologous to each other typically remain hybridized to each other. Such stringent conditions are known to those skilled in the art and can be found in, for example, Current Protocols in Molecular Biology, John Wiley & Sons, N.Y. (1989-2006). One example of stringent hybridization conditions is hybridization in 6× sodium chloride/sodium citrate (SSC) at about 45° C., followed by one or more washes in 0.2×SSC, 0.1% SDS at 50-65° C.

Biomarker Detection in Cancer by MPAT.

mAb BF819 enables detection of BF819 expression in biological samples, and patient test samples, particularly detection in protein samples from normal and disease human specimens, where the disease is cancer, or from cancer tissue or cell lines, thus correlating BF819 expression with the presence of cancer.

Matrix Protein Array Technology.

Assessment of differential expression of BF819 includes immunodetection, and specifically using mAb BF819 in a process named the Matrix Protein Array Technology (MPAT) as presented below and described in detail in Example 3.

The MPAT is a multiplex protein array immunoassay that simultaneously analyzes multiple biological samples. In essence, the MPAT is an immunoassay linked to a data acquisition and imaging system, whereby the same matrix of samples is simultaneously interrogated by an antibody. Then, a secondary antibody, preferably linked to a chemiluminescent probe or fluorescent dye, is used to visualize antigen-antibody reaction for each sample, and a scanned image of all reactions is produced with an imaging system, processed and analyzed to yield the simultaneous examination in multiplex format of the relative expression levels of a number of proteins of interest.

The solid support of the matrix protein array is preferably nitrocellulose or glass, yet can be made of a variety of materials that include, but are not limited to: plastic, polystyrene, nylon, teflon, ceramic, fiber optic and semiconductor materials. The solid support of the matrix protein array is composed of different physical areas that can be referred to as wells, compartments, surfaces, and the like, distinctly separated from each other. These physical areas can adopt a variety of surfaces and volumes, and the support can accommodate from 1 or 2 to more than 10,000 compartments, depending on the needs, leading to an extremely versatile tool. Each compartment may contain biological samples from the same type, different types, the same species, different species, the same physiological condition, different physiological conditions or any combination of the above arrayed on the solid support. Each compartment is overlaid with any identifier, preferably an antibody, as selected.

It is understood by those skilled in the art that the device and methodology described herein as MPAT allows all kind of combination of biological samples, number of samples, conditions of samples, size of compartment of the matrix protein arrays, type of identifiers, or any permutation of the above. Furthermore, while in the present invention, the MPAT methodology described below is applied to human biological samples, it is understood to the skilled in the art that the MPAT is widely applicable to protein samples derived from any organism, source, including animal, bacterium, yeast, fungus, or plant.

In its simplest format, the MPAT is composed of 96 chambers although other formats can be used depending on the number of antibodies to assay and the number of samples to screen. In a given MPAT experiment, the same matrix of protein extracts from different biological samples (e.g. clinical specimens or cancer cell lines as described below) is printed in each chamber, and each chamber is assayed with a distinct individual antibody. Each individual compartment is then overlaid with a distinct antibody and processed for the detection of antigen-antibody complexes. This format allows direct comparison between multiple samples (including normal and diseased samples) under the same conditions, preventing day-to-day experimental variability, as it is often observed in other proteomic studies (Diamandis E P, Analysis of serum proteomic patterns for early cancer diagnosis: drawing attention to potential problems, Natl Cancer Inst 96:353-356, 2004a; Diamandis E P, Mass Spectrometry as a diagnostic and cancer biomarker discovery tool, Mol Cell Proteomics 3:367-378, 2004b; Ransohoff D F, Rules of evidence for cancer molecular-marker discovery and validation, Nature Rev Cancer 4: 309-314, 2004) or DNA microarray experiments (Dudoit S, Gentleman R C, Quackenbusch J, Open source software for the analysis of microarray data, Biotechniques 34:S45-S51, 2003; Gabor Miklos G L and Maleszka R, Microarray reality checks in the context of a complex disease, Nature Biotechnol 22:615-618, 2004).

In the immunodetection analysis detailed in Example 3 and described in a number of preferred embodiments herein, the detection and isolation of the marker disclosed herein from within a complex biological mixture (i.e. antibody-antigen complexes) is preferably performed by way of a chemiluminescent reaction, although other protocols based on other labeling and detection systems, such as alkaline-phosphatase, biotin-streptavidine, and fluorescence can also be successfully used within the scope of the present invention. Antigen-antibody signals are captured by a charge-coupled device (CCD-camera) or a Li-cor-Odyssey infrared imaging system, processed and quantified by specialized software, as described in Example 3.

Differential Expression of BF819 in Cancer Versus Normal.

The differential expression of BF819 in patient tissue samples (specifically in protein extracts thereof), including cancer, normal, and benign, using the MPAT technology demonstrates the utility of BF819 as a marker for cancer detection.

Five independent MPAT studies were performed with mAb BF819, as described in Example 4, using four different sets of clinical samples. First mAb BF819 was used to detect the presence of BF819 in 213 clinical samples, including protein extracts from breast, colon and lung normal, cancer and benign tissues. This first experiment indicated that BF819 is overexpressed in cancer versus normal. Second, to confirm this result, mAb BF819 was assayed on 741 samples, exclusively comprising breast and colon normal, cancer and benign patient tissue extracts. Third, the analysis was escalated to 1329 clinical samples including normal, cancer and benign samples from breast, colon, lung and ovary. At this point, BF819 was confirmed to display differential expression in cancer versus normal samples, and mAb BF819 was subcloned. The 1329 sample study was repeated with subcloned mAb BF819 to ensure that the subcloned mAb had the same reactivity and properties as the original mAb. Finally, subcloned mAb BF819 was tested on the largest array of 1471 clinical samples.

Composition of Clinical Sample Sets.

Cancer samples used in these experiments encompass the three major cancers in terms of incidence in the US, as colon, breast and lung cancers are represented (Siegel R 2012).

Table 1 summarizes the composition of the clinical sample sets, listing organ site, the total number and type of specimens, the number of normal, benign and cancer samples, including early and late stage patients. Normal samples in these experiments include normal breast, colon, lung and ovary tissues (e.g. normal breast from typical macromastia cases) as well as “normal adjacent to tumor” (NAT), i.e. normal tissue deriving from the same patient from which the tumor tissue is derived. NAT are preferred normal controls as they allow taking into account individual patient variations.

The 213 tissue sample experiment included: 32 breast cancer, 16 breast normal and 7 benign; 64 colon cancer, 26 colon normal and NAT, and 20 benign; 32 lung cancer and 16 lung NAT, amounting to 128 cancer, 58 normal and NAT, and 27 benign samples (Table 1). The 741 tissue sample experiment included: 115 breast cancer, and 175 breast NAT and 15 benign; 173 colon cancer, and 240 colon NAT and 23 benign, amounting to 288 cancer, 415 NAT and 38 benign samples (Table 1). The 1329 tissue sample experiment included the following samples: 115 breast cancer, 175 breast NAT and 15 benign; 173 colon cancer, 240 colon NAT and 23 benign, 199 lung cancer, 208 lung NAT and 15 benign; 88 ovarian cancer, 43 ovarian NAT and 35 benign, amounting to 575 cancer, 666 NAT and 88 benign samples (Table 1). The 1471 tissue samples experiment included the following samples: 116 breast cancer, 138 breast normal and NAT, and 22 benign; 198 colon cancer, 284 colon normal and NAT, and 17 benign; 186 lung cancer, 233 lung normal and NAT, and 14 benign; 123 ovarian cancer, 78 ovarian normal and NAT, and 62 benign, amounting to a total of 623 cancers, 733 normal and NAT, and 115 benign samples (Table 1).

Whenever possible, an almost equal number of early and late stages of cancer samples were included at each organ site, with “early” including stages I and II, and “late” including stages III and IV. The 1329 clinical sample set featured: 59 early and 56 late stage breast cancers, 89 early and 84 late stage colon cancers, 156 early and 43 late stage lung cancers, and 36 early and 52 late stage ovary cancers. On the other hand, the 1471 clinical sample set featured: 49 early and 67 late stage breast cancers, 95 early and 103 late stage colon cancers, 141 early and 45 late stage lung cancers, and 36 early and 87 late stage ovary cancers.

While most breast cancers are ductal carcinomas, and most colorectal cancers are adenocarcinomas, lung and ovary cancers present with different histological subtypes. Accordingly, the clinical sample sets used herein reflect this epidemiological evidence.

There are different types of lung cancers (Brambilla, 2001; ACS 2012): the two major ones are small cell lung cancer (SCLC) and non-small cell lung cancer (NSCLC). NSCLC is the most common type of lung cancer, while SCLC accounts for 20-25% of all lung cancers, a clinically important distinction, as SCLC is more responsive to chemotherapy than NSCLC. NSCLC in turn comprises several distinct histologies including: adenocarcinoma, squamous cell carcinoma, large cell carcinoma, bronchio-alveolar cell carcinoma (BAC) and others. Adenocarcinoma and squamous cell carcinoma represent the major subtypes within NSCLC.

In the 1329 sample set, the 156 early stage lung cancer samples comprised: 55 adenocarcinoma, 47 squamous, 15 large cell carcinoma, 10 bronchioalveolar, 2 small cell lung cancer, 13 non small cell lung cancer and 14 other minor types; while the 43 late stage lung cancer samples comprised: 19 adenocarcinoma, 18 squamous carcinoma, 2 large cell carcinoma, 2 non small cell lung cancer, and 2 mixed. Hence, out of 199 total lung cancer samples, the set featured: 74 adenocarcinoma, 65 squamous carcinoma, 17 large cell carcinoma, 15 non small cell carcinoma, and 15 other minor types, where adenocarcinoma and squamous carcinoma represent the major subtypes like in the actual patient population.

In the 1471 sample set, the 141 early stage lung cancer samples comprised: 66 adenocarcinoma, 51 squamous, 17 large cell carcinoma, and 7 non small cell lung cancer; while the 45 late stage lung cancer samples comprised: 21 adenocarcinoma, 20 squamous carcinoma, 2 large cell carcinoma and 2 non small cell lung cancer. Hence, out of 186 total lung cancer samples, the set featured: 87 adenocarcinoma, 71 squamous carcinoma, 19 large cell carcinoma, and 9 non small cell carcinoma, where adenocarcinoma and squamous carcinoma represent the major subtypes like in the actual patient population.

Epithelial ovarian carcinomas represent 85% of all ovary cancers, and comprise 4 major subtypes: serous (50%), endometrioid (10-25%), mucinous (10-15%), and clear cell type (5%; ACS, 2012).

In the 1329 sample set, the 36 early stage ovary cancer comprised: 8 serous papillary adenocarcinoma, 10 mucinous, 10 endometroid, 4 clear cell type, and 4 mixed; and the 52 late stage ovary cancers comprised: 30 serous, 7 mucinous, 8 endometroid, 1 clear cell, 6 mixed. Hence out of 88 total ovary cancer samples, the set featured: 38 serous (43%), 17 mucinous (19%), 18 endometroid (20%), and 5 clear cell types (11%), a representation compatible with the distribution of ovarian cancer subtypes in the actual patient population.

In the 1471 sample set, the 36 early stage ovary cancer comprised: 13 serous papillary adenocarcinoma, 10 mucinous, 9 endometroid, and 4 clear cell type; and the 87 late stage ovary cancers comprised: 76 serous, 2 mucinous, 9 endometroid. Hence out of 123 total ovary cancer samples, the set featured: 89 serous (72%), 12 mucinous (9.7%), 18 endometroid (14.6%), and 4 clear cell types (3%), representing proportions similar to the actual patient population.

Benign samples in the 1329 sample set comprise: 15 in breast, mostly including fibroadenoma; 23 in colon, including adenomatous polyps and tubulovillus adenoma; 15 in lung, including solitary fibrous tumor and hamartoma; and 35 in ovary (including 5 serous cystadenofibroma, 3 serous cystadenoma, 10 mucinous cystadenoma, 2 cystadenomafibroma, 3 benign cysts, 5 mixed benign and 3 fibroma, 4 fibrothecoma).

Benign samples in the 1471 sample set comprise: 22 in breast, all fibroadenoma; 17 in colon, including adenomatous polyps and tubulovillus adenoma; 14 in lung, including solitary fibrous tumor and hamartoma; and 62 in ovary (including 33 serous cystadenoma, 9 mucinous cystadenoma, 8 cystadenofibroma, 5 benign cysts, 4 mixed benign and 3 fibrothecoma). Preparation of tissue protein extracts is described in Example 4.

Overexpression of BF819 in Cancer.

FIG. 3 illustrates the results obtained with subcloned mAb BF819 in the 1329 sample experiment. Background level of reaction is observed with normal samples, both in the lung and ovary subset. In contrast, as evidenced in FIG. 3, BF819 is overexpressed in lung and ovary cancer versus normal samples, with 51-62% of colon and lung cancer samples respectively, up to 72% of ovary cancer samples reacting in this experiment and clinical sample set, indicating high prevalence of the marker.

Moreover, difference in expression of BF819 is not only observed in late stage of cancer, but also in early stage of cancer, at both organ sites. In particular, about 45-58% respectively of early stage colon and lung cancer samples, and up to 67% of early stage (versus 75% of late stage) ovary cancer samples are detected by mAb BF819 in this experiment and clinical sample set. These results establish that Bm BF819 has diagnostic utility in the early detection of colon, lung and ovarian cancers.

Some mAb BF819 reactivity is detected in colon and ovary benign versus normal samples in a limited sample set (23). No particular overexpression of BF819 is detected in lung benign versus normal control. Reactivity of mAb BF819 in these clinical samples establishes the utility of BF819 as a marker, particularly for lung, colon and ovarian cancer.

CEA Detection in Cancer Tissues by MPAT.

To further investigate the difference between BF819 and CEA, the standard in colon cancer detection, an MPAT experiment was performed on the 1471 clinical sample set using a commercial antibody against CEA.

Carcinoembryonic antigen (CEA) was first identified as an antigen present in both fetal colon and colon adenocarcinoma but that appeared to be absent from healthy adult colon (reviewed in Duffy, 2001). CEA has been the most thoroughly investigated marker in colorectal cancer. It is recognized that CEA has no clinical utility as screening or early detection marker because most patients will present with CEA-negative disease at time of diagnosis (Sturgeon, 2002), and because CEA may be elevated in liver diseases and other cancers (Duffy, 2001). Rather (as reviewed in Locker 2006; Brunner, 2009), CEA is in clinical use for determining prognosis, monitoring colorectal cancer progression and recurrence, monitoring therapy in advanced disease, and in disease surveillance following curative resection.

As illustrated in FIG. 4, a control experiment was performed on the larger clinical set of 1471 samples. CEA is essentially not detected in breast, lung and ovary tissues, whether in cancer, benign or normal samples, with only few scattered positive reactive spots (i.e.: 1 breast normal, 2 breast cancer; 3 lung cancers, 3 ovary cancers). In contrast, some CEA expression is detected in some colon cancer samples (limited to about 20 of the 200 colon cancer spots), significantly less than the overexpression of BF819 observed throughout the majority of colon cancer versus normal. Comparing the reactivity of BF819 whether in lung or ovary cancer samples, in both early and late stage, to that of CEA in both early and late stage colon cancer samples (compare FIG. 4 to FIG. 3) BF819 reacts significantly more than CEA not only on late stage colon cancer tissues, but also on early stage colon cancer tissues. BF819 thus appears consistently prevalent in both early and late stage cancer performing as a better colon tissue biomarker than CEA, and the data demonstrate that BF819 has superior performance in detection of both early and late stage cancer, and for colon, lung and ovarian cancers, compared to CEA for colon cancer.

BF819 Expression in Cancer Cell Lines.

As described in details in Example 5, equal amounts of protein extracts from 28 different cancer cell lines are spotted on the MPAT, and assayed with mAb BF819. The cell lines are derived from breast, lung, colon, ovary and prostate cancers, as well as from melanoma, hepatocarcinoma, lymphoma, and glioblastoma.

As evidenced in FIG. 5, mAb BF819 reacts with total protein extracts from most colon (3/4) and lung (3/6) cancer cell lines, and from the ES2 ovary cancer cell line, consistent with data obtained with patient tissue extracts by MPAT (FIG. 3). It also strongly reacts with some of the breast and other (derived from prostate, hepatic, lung, colon, ovarian cancer) cell lines and all melanoma cell lines tested. mAb BF819 also yields a positive reaction with protein extracts derived from lymphoma, human B lymphoblast, brain glioblastoma, and oropharyngeal epdermoid carcinoma cell lines, but react poorly or not at all with both hepatic carcinoma.

While cancer cell lines are not always exactly representative of overexpression in cancer tissue, the study of BF819 expression in the 28 cancer cell lines confirms and expands on the MPAT data obtained using tissue extracts from patient clinical samples (see above, FIG. 3), whereby BF819 is overexpressed in colon, lung and ovary cancer versus normal controls. These results also suggest that BF819 may be overexpressed in breast cancer as well as in prostate cancer and melanoma, for which tissue samples are scarce.

The reactivity of mAb BF819 in 28 cancer cell lines, both confirms and extends to other cancers, the presence, expression and diagnostic utility of BF819 as a marker as previously demonstrated in colon, lung and ovary patient tissues.

BF819 Expression in Tissue by Immunohistochemistry.

Immunohistochemistry (IHC) is a commonly practiced in vitro diagnostic procedure to determine normal vs. disease in a patient tissue biopsy. The patient tissue biopsy is first formalin-fixed and paraffin-embedded, then sectioned at 3-5 micrometer thick and mounted on treated microscope glass slides to enhance tissue adherence. Slides are stained with a relevant antibody against a cellular marker in a procedure described in details in Example 6. Tissue microarrays (TMA) can also be used instead of individual slides to analyze the reactivity of an antibody, or marker expression, in a large number of patient samples to establish marker prevalence in a biological sample of the patient.

The association of BF819 to cancer is further evidenced by IHC using mAb BF819 and tissue slides featuring cancer tissues and normal controls, preferably adjacent normal controls (NAT above). The immunostaining procedure comprises the use of anti-mouse IgG biotinylated secondary antibody followed by streptavidin linked to horseradish peroxidase, finally followed by the addition of AEC substrate. Other immunostain procedures are contemplated: for example, protocols based on different labeling and detection systems, such as alkaline-phosphatase, biotin-streptavidine, or fluorophores can also be successfully performed within the scope of the present invention. Furthermore, while most tissues undergo pre-treatment to inactivate endogenous peroxidase, if peroxidase-based staining is used, pre-treatment is not necessary when using fluorescence-based imaging system.

Proteins have different localization within the cell depending on their function, including secreted (such as growth factors, hormones, neuropeptides), present on the cell surface (such as glycoproteins, glycolipids and receptors) intracellular (within the cytosol, or in particular sub-cell compartments, such as the nucleus, the Golgi, or the endoplasmic reticulum). BF819 can be localized to cellular structures via the use of mAb BF819 by a variety of techniques known to those skilled in the art, which can be performed on mammalian cell suspension or adherent cells, and which are described in (Current Protocols in Immunology, Wiley Interscience, John E. Colligan et al.), such as but not limited to, immunohistochemistry, immunfluorescence (IF) using FACScan (FACS), flow cytometry (FC) and indirect IF, but also electron microscopy and other imaging techniques providing localization to subcellular structures. By IHC, specific staining of mAb BF819 against marker BF819 can be localized to either nuclei, cell membrane or cytosol.

Knowledge of biomarker localization is important in diagnostic applications. mAb BF819 was used to detect the presence and localization of BF819 in patient tissues by IHC, as described in Example 6 (data not shown). Different tissue biopsies from patients with different cancers were tested. BF819 was found in most cancers, including breast, colon, lung, ovary and prostate cancer. mAb BF819 specifically stained tumor versus normal tissues from the same patient, with cytoplasmic and nuclear staining of most cancer cells. This result indicates that BF819 is an antigen that can be found in the cell nucleus. BF819 was also found in a pancreatic tumor in the omentum.

The IHC data presented herein further confirm the presence of BF819 in colon, lung and ovary cancers, as already demonstrated in patient tissue extracts by MPAT (FIG. 3). In addition, the IHC data demonstrate that BF819 is also expressed in breast, colon and prostate cancer tissues, as suggested form the cancer cell line experiment (FIG. 5), thus expanding on previous data. Finally, the IHC data presented herein also demonstrate the presence of BF819 in pancreatic cancer tissues, as further confirmed below. The experiments in patient tissue extracts and in cancer cell line protein extracts by MPAT, and in patient tissues by IHC, confirm the diagnostic utility of BF819 and mAb BF819 in cancer, with particular emphasis on the use of IHC in in vitro cancer diagnostics.

BF819 Immunodetection by Western Blot.

In another embodiment of the invention, BF819 is further analyzed and characterized by Western blot, as described in details in Example 7, and illustrated in FIG. 6. Protein extracts from human cancer cell lines, or protein extracts derived from matched or unmatched normal and tumor tissue samples from cancer patients can be used to detect BF819. Protein extracts are separated by gel electrophoresis, transferred to nitrocellulose and probed with mAb BF819 to visualize the corresponding antigen protein band. Based on antibody-antigen reactivity in cancer cell lines or in normal and disease tissues, BF819 is shown to be expressed or overexpressed in cancer. Furthermore, Western blot analysis provides an apparent molecular weight for BF819, confirming amino acid and nucleotide sequence data and the identity of BF819 (See FIGS. 1, 2, and 7.)

As evidenced in FIG. 6, mAb BF819 detects two major protein bands of an apparent molecular weight of 15 and 32 kDa respectively, in all protein extracts from all cancer cell lines tested, including colon and breast cancer cell line mix (FIG. 6, lane C/B), a prostate and lung cancer cell line mix (lane P/L) and an ovary and lung cancer cell line mix (lane O/L). Predominance of the immunodetected bands may vary in each lane, depending on the extract.

These results confirm the expression of BF819 in breast and colon, lung, ovary and prostate cancer, in accordance with data obtained above by MPAT, whether in patient tissue extracts (FIG. 3), in cancer cell line extracts (FIG. 5), or in cancer tissue biopsies by IHC (data not shown).

As small variations between the observed and expected MW of a protein band in Western blot are known to the skilled in the arts, the observed protein band of 32 kDa is consistent with Bm BF819 expected MW of 28,377 Da. While other isoforms have not been described for CTDNEP1 (RefSeq; Pruitt, 2012) potential cancer-related variants, may yield additional isoforms accounting for the 15 kDa observed protein band.

BF819 Secretion in Cancer Cell Line Tissue Culture Supernatants.

To explore whether BF819 is secreted in patient body fluids, mAb BF819 is used to detect BF819 in tissue culture supernatants of cancer cell lines, as described in detail in Example 8. 12 different cancer cell lines from breast, colon, lung, ovarian and prostate cancers, as well as normal embryonic fibroblasts as control, were grown in medium without fetal calf serum to facilitate the detection of potentially secreted biomarkers (as the presence of serum albumin in fetal calf serum may hinder detection of secreted proteins). Equal amounts of protein extracts from said tissue culture supernatants were spotted on the MPAT, and reacted with mAb BF819, followed by detection of antigen-antibody complexes.

As evidenced in FIG. 7, mAb BF819 strongly reacts with secreted proteins present in the culture supernatants of lung (NCI-H1792 and to a lesser extent NCI-H157), colon (COLO 320), ovary (ES2), and the prostate (PC-3) cancer cell lines. No reactivity is detected between mAb BF819 and potentially secreted proteins from other cancer cell lines or NEF control.

As further illustrated in FIG. 8, expression and secretion of BF819 were also tested in four cervical cancer cell lines. BF819 was strongly expressed in extracts from the HPV-containing Ca Ski, ME-180 and SiHa cervical cancer cell lines, and to a lesser extent from the non-HPV containing C-33A cell line (FIG. 8, lane x). Some secretion was observed in cell culture supernatant without fetal calf serum (lane s−) of HPV containing Ca Ski and SiHa (FIG. 8, lane s). This experiment demonstrates that BF819 is secreted in the culture supernatant of cervical, colon, lung, ovary and prostate cancer cell lines, suggesting that BF819 may be further secreted in patient body fluids.

BF819 Secretion in Biological Fluids: Serum.

BF819 is secreted in the supernatant of several cancer cell lines, thereby suggesting secretion in patient body fluids. Examples of patient test samples include, but are not limited to, blood, lymph, serum, plasma, urine, gynecological fluids and smears, bronchio-alveolar lavages, sputum, nipple aspirate fluids, etc. In many instances, such samples are associated with the detection of diseases and conditions at specific organ sites, e.g. bronchio-alveolar lavages for asthma, lung cancer and lung diseases, nipple aspirate fluids for breast cancer, urine sediments after digital rectal examination for prostate cancer, etc. Among body fluids, serum and urine are particularly important, as they represent an informative biological material not requiring invasive procedures.

BF819 is also demonstrated to be present in the serum of pancreatic cancer patients versus controls (normal individuals as well as patients with benign and inflammatory conditions), using mAb BF819 via MPAT, as described in details in Example 9 (data not shown).

To this end, a set of 165 serum samples was pulled from a broad cancer collection of over 2,000 serum specimens, and comprised: 91 pancreatic cancer cases (PaC), 16 benign tumors of the pancreas (BN), and 14 chronic pancreatitis cases (IFN), i.e. the major inflammatory condition of the pancreas that traditionally poses a diagnostic dilemma in pancreatic cancer differential diagnosis, and 44 normal controls (NL) as summarized in Table 2. The PaC cases were exclusively ductal adenocarcinoma of the pancreas, as it represents 85% of all pancreatic cancers and the most aggressive one, and included 50 early stage and 36 late stage, while 7 had no stage information. The 16 BN included the following cases: tubulovillous adenoma (1), mucinous cystadenoma (3), mucinous cyst (1), serous cystadenoma (6), intraductal papillary mucinous neoplasm (IPMN, 1) and other cysts (3).

Based on the MPAT experiment (data not shown) and its statistical analysis summarized in Table 3, BF819 is overexpressed in PaC versus non-PaC serum samples (BN, IFN and NL). Indeed, as indicated in Table 3 for the “late stage PaC group versus the non-PaC group” comparison, BF819 shows good discriminatory power yielding AUC values of 0.70 with strong significance (p<0.0005). In contrast, no significant difference is found when comparing the same groups in the negative control case. The “no primary antibody control” uses the same matrix of serum samples reacting with buffer alone instead of the mAb of the present invention, followed by the secondary antibody. This control is meant to reveal potential background due to non-specific binding of the secondary antibody. Indeed, as shown in Table 3, a AUC value of 0.52 and a 95% CI including 0.5 chance line, and p values>0.05 are all consistent with a good negative control, strengthening the diagnostic utility of BF819. Thus, the overexpression of BF819 in late stage PaC versus non-PaC controls is statistically significant, indicating clinical application in diagnostics.

The data disclosed demonstrate that BF819 is overexpressed in the serum of pancreatic cancer patients versus controls in a statistically significant manner, confirming the presence of BF819 in biopsied tissues from pancreatic cancer patients as determined by IHC.

BF819 Secretion in Biological Fluids: Urine.

BF819 is secrected in the supernatant of some cancer cell lines and in the serum of patients with pancreatic cancer, strongly suggesting, that BF819 is secreted in other patient body fluids. Among body fluids, serum and urine are particularly important, as they represent an informative biological material not requiring invasive procedures. In a preferred embodiment of the present invention, BF819, a fragment thereof, or a BF819 epitope is also detected in the urine of cancer patients versus normal controls, using mAb BF819 and the MPAT, as described in detail in Example 10.

Two methods of urine sample preparation were used. Because, urine has low protein abundance, marker concentrations are enhanced by acetone-precipitating the proteins, as described in Example 10. Protein pellets were then resuspended in Tris buffer with Triton, homogenized and their concentration measured. Equal amounts of proteins were printed on the MPAT membrane in a double-blind experiment (“precipitated urine”). Urine samples were spotted “as is” on the MPAT membrane, also in a double-blind experiment (“unprecipitated urine”).

A large clinical set (n=305) was used in the case of precipitated urine samples, while a small set (n=47) was used in the case of unprecipitated urine, as described in Example 10, and summarized in Table 4. As indicated in FIG. 9 legend and Table 4, a variety of cancer patients were used in this experiment. Considering the likely possibility of prostate and kidney cells from prostate and kidney cancer patients shedding into urine, a significant number of urine samples from prostate cancer patients (n=107), representing stages II to IV, and from some kidney cancer patients (n=6) of stage I to III were included. Considering that BF819 is overexpressed in colon cancer tissues and detected in colon cancer cell extracts and patient tissues, 35 samples from colon cancer patients, from stage I to IV were included, as well as samples from patients with benign colon disease (n=10) and inflammatory conditions of the colon (n=32), in addition to 92 normal controls. Finally, because BF819 was found secreted in the serum of pancreatic cancer patients, urine samples from 21 cases of pancreatic cancer and 1 benign pancreatic tumor were tested.

As illustrated in FIG. 9, while reactivity is observed in normal individuals, mAb BF819 clearly reacts with precipitated urine proteins in all disease groups. In this experiment and clinical sample set, mAb BF819 detects BF819, or a fragment thereof, in the precipitated protein fraction of the urine specimens of colon cancer cases, and to a lower extent in the urine specimens of colon inflammation and colon benign cases, ranging approximately from 30% (colon benign) to 43% (colon cancer). Similarly to results in colon cancer tissues by MPAT (FIG. 3), BF819 or a fragment thereof is detected both in early and in later stages of cancer in the urine specimens of these patients.

Furthermore, in this experiment and clinical sample set, mAb BF819 detects BF819, or a fragment thereof containing the BF819 epitope, in 20% of precipitated urine samples from prostate cancer patients, including early stage (FIG. 9, panel B: spots H10, H11, H21, I1, I6, I7, I10, I17), as well as late stage (spots J11, J13, J16, J17, L4, L9, L19) prostate cancer.

Strong reactivity is also detected in 1 of 6 kidney cancer and in the kidney benign case (F20). Pathology reports related to the latter indicates that it is an “epithelial lesion consistent with nephrogenic rest (embryonic tissue)”. As known in the art, embryonic expression patterns and proteins are often found expressed in cancer, which could explain detection of the BF819 epitope.

Significantly, BF819 reactivity is also found in the urine of up to 8 pancreatic cancer patients (38%), almost all early cases: A1, A2 (stage II), A10, A11, (stage I), A1, A13, A17 (stage II), and A21 (unknown stage). No reactivity is found in the urine of the benign pancreatic patient (A22). The results in urine confirm that BF819 is found in pancreatic cancer patient tissues as determined by IHC (data not shown) as well as in pancreatic cancer patient serum (Table 3).

Detection of CEA and PSA, or fragments thereof, was attempted in the same precipitated urine samples used in the experiment above (FIG. 11), from the same cancer patients (Table 2) and in the same experimental conditions (Example 9). As shown in FIGS. 12 and 13, using commercially available antibodies against CEA and against PSA, neither CEA (FIG. 12) nor PSA (FIG. 13), nor fragments thereof are detected in these conditions in the urine of any of the relevant cancer patients, benign, inflammatory or normal controls. CEA and PSA are the current standard for colon cancer and prostate cancer, respectively. Both markers are also extensively used in immunohistochemistry to confirm colon cancer and prostate cancer in tissue biopsies.

These data show that mAb BF819 detects the BF819 epitope (SEQ ID NO: 3) found in the precipitated protein fraction of urine samples from cancer patients, particularly in colon, pancreatic and prostate cancers. The results of the experiment using precipitated urine samples described above (FIG. 9) are further confirmed by the results obtained in unprecipitated urine samples. As illustrated in FIG. 12, mAb BF819 reacts with unprecipitated urine samples from patients with pancreatic cancer, colon cancer and inflammation, and prostate cancer, while no reactivity at all is observed in any of the normal controls or colon benign case.

The number of samples in the unprecipitated urine sample experiment is limited. However, the reactivity of mAb BF819 against the BF819 epitope present in the urine of cancer patients appears improved in the unprecipitated urine sample experiment with respect to the precipitated sample experiment.

In particular, while reactivity was 38%, 43%, 37%, and 20% respectively in pancreatic cancer, colon cancer, colon inflammation and prostate cancer cases when urine specimens were precipitated, reactivity increased to 60% for pancreatic, colon cancer and inflammation cases, and up to 83% for prostate cancer cases when urine specimens were unprecipitated. Note that colon benign and normal cases remained negative. The increase in sensitivity of detection in prostate cancer is particularly revealed by diluting unprecipitated urine samples in 1:2 Tris-Triton buffer. Reactivity in all disease groups is decreased when samples are diluted in 1:10 in the same buffer.

Note that the urine specimens in this experiment are derived from patients with early stage of cancer according to their pathology report. Stages I and II are defined as early, while stages III and IV are defined as advanced and late. Indeed, all colon cancer specimens are either stage I or stage II; all pancreatic cancer specimens are stage II, with A13 of unknown stage, and most prostate cancer samples are stage II.

The data disclosed herein show that mAb BF819 detects BF819, or a fragment thereof displaying the BF819 epitope in the urine of colon, pancreatic, and prostate cancer patients, in early and late stage, and to a certain extent in the urine of kidney cancer patients (FIGS. 9 and 12). Bm BF819 or a fragment thereof is thus secreted in urine. In contrast, no detection of CEA or PSA is observed in the same conditions.

In conclusion, the data disclosed demonstrate that BF819 is found in tissues from breast, colon, lung, ovary, pancreatic and prostate cancer patients as determined by IHC, and is overexpressed in tissue extracts from colon, lung and ovary cancer patients versus normal controls, including in early stages, as demonstrated by MPAT (FIG. 3). Furthermore BF819 is found in the serum of pancreatic cancer patients and secreted in the urine of colon, pancreatic and prostate cancer patients, including at early stage (FIGS. 9 and 12).

### EXAMPLES

The following abbreviations are used throughout. hr: hour; min: minutes; sec: seconds; rpm: rotation per minute; RT: room temperature; ON: overnight; Bm: biomarker; mAb: monoclonal antibody.

### Example 1

### Epitope Analysis by Phage Display

The amino acid sequence within the marker of the present invention that is recognized by the mAb is defined as the “epitope”. To find the epitope harbored by the marker of the present invention, a phage display approach was employed using the New England Biolabs PhD-12 Phage Display Library Kit according to the manufacturer's instruction manual. A brief description of the protocol follows.

The phage library has a titer of 10¹³ pfu/ml. Ten microliters of the library are incubated in TBST (50 mM Tris-HCl pH 7.5, 150 mM NaCl, 0.1% Tween 20) with the mAb BF819 for 1 hr at RT. The mAb is immobilized on a plastic surface, such as an ELISA 96 well plate, via a rabbit mouse IgG (see below). This represents a ˜10¹¹ pfu input, i.e. a ˜100 fold representation of a library with a complexity of 10⁹ individual clones, each harboring five copies of a 12-mer peptide embedded in the phage capsid. After incubation and washing of unbound phages in TBST, bound phages are eluted in 0.2 M glycine-HCl pH 2.2, 1 mg/ml BSA, then neutralized in 1 M Tris-HCl pH 9.1 and finally amplified in the appropriate bacterial strain (ER2731) by growing for 4-5 hrs at 37° C. with vigorous shaking. Phage is collected upon removal of bacterial cells by centrifugation, and precipitated by 20% PEG in 2.5 M NaCl at 4° C. ON. Phage is titered in order to carry out a second and third panning with an input titer equivalent to that of the first round. Note that stringency increases from the first to the third panning, by increasing Tween 20 concentration (0.1% to 0.5%) and decreasing incubation time (1 hr to 30 min).

To decrease non-specific binding of the phage library, the three panning steps are first preceded by a pre-panning (prior to incubation of phage library with the relevant mAb), whereby the phage library is incubated with non-specific or pre-immune mouse IgG immobilized to 96 well plates via a rabbit anti-mouse IgG. This pre-panning incubation step eliminates all phages in the library that would non-specifically bind to plastic, rabbit anti-mouse IgG and mouse IgG. Rabbit anti-mouse IgG plate coating is used to concentrate the relevant mAb (or the mouse IgG in the pre-panning step), which is in the form of hybridoma culture supernatant rather than in the form of purified mAb; such coating is also used to enhance mAb binding to the plate. Pre-panning is carried out ON at 4° C. or 1 hr at 37° C.

Upon the three cycles of panning and amplification, an enriched population of phage is collected; this enriched population harbors, within the capsid amino acid sequence, those 12-mer peptides that are specifically bound by mAb BF819 and thus represent, at least in part, the epitope of the protein marker BF819.

After the third round of panning, resulting phages are titered (yet not amplified), and about 20 individual phage plaques are picked, amplified in a small volume of bacterial culture, and phage DNA is prepared upon a simple phenol chloroform extraction followed by ethanol precipitation. Phage DNAs are sequenced and the 12-mer peptide amino acid sequence is determined for each clone. Comparison of the amino acid sequences from the 20 clones eventually yields a consensus epitope sequence, usually a 5-6 amino acid core within the 12-mer, that is recognized by the mAb BF819.

Non-redundant individual phages (i.e. with different 12-mer sequences) are amplified in medium sized cultures and titered in order to be further assayed by direct ELISA in the presence of mAb BF819. This ELISA assay will identify the clone(s) harboring the best 12-mer binders. Comparison of the best binder sequences in turn provides the consensus epitope of mAb BF819.

Briefly, ELISA plates are first coated with the mAb of the present invention (10-100 μg/ml, diluted when necessary in 0.1 M NaHCO3 pH 8.6) by ON incubation at 4° C., or 1 hr at 37° C. Each step is followed by 10 TBST washes (0.5% Tween 20). Plate wells are then blocked with blocking solution (see above) for 1-2 hr at 4° C. Serial four-fold dilutions of phage to be tested (from 1012 to 2×105 virions per well) are prepared in a separate pre-blocked 96 well plate in TBST (0.5% Tween 20), and added to mAb BF819 coated wells, and to the uncoated (but blocked) wells used as negative controls, for 1-2 hr incubation at RT with agitation. Phage-mAb complexes are detected by colorimetric reaction at 450 nm involving a 2-step process: i) 1 hr incubation at RT with horse radish peroxidase (HRP) conjugated anti-M13 antibody according to manufacturer's instructions (GE Healthcare), followed by ii) addition of TMB (HRP substrate) until a blue color develops. Signal intensities are compared to the “no mAb” control, and the reaction stopped by H₂SO₄.

### Example 2

### Determination of Marker BF819 Identity

The consensus epitope of mAb BF819 of the present invention, as determined from the phage display approach (Example 1 above), must be present in the amino acid sequence of the protein marker.

Thus, the protein identity BF819 is determined upon BLAST search of the consensus epitope in the NCBI protein database. Specifically, the consensus epitope or alternatively the 12-mer peptide with the best ELISA binding is entered in a BLAST search (blast.ncbi.nlm.nih.gov) to retrieve all possible proteins with a degree of homology to the consensus epitope or queried peptide of up to 80%.

Bm identity and its protein sequence is determined upon correlation with other specific marker data presented herein (western blot, molecular weight, subcellular localization, biomarker expression by IHC etc.) and other databases, such as Human Protein Atlas (www.proteinatlas.org) and UniProt database (www.uniprot.org).

### Example 3

### Matrix Protein Array Screening Technology

The matrix protein array technology (MPAT) is a multiplex protein array immunoassay developed by the Applicant for the simultaneous analysis of multiple biological samples, under the same conditions. The MPAT has been used for the immunodetection of protein marker/mAb of the present invention in a variety of protein samples, as detailed in the examples below.

The solid support of the matrix protein array may be composed of a different number of chambers or compartments of different sizes, depending on the scope of the investigation. In its simplest format, the MPAT is composed of 96 chambers. Other formats can be used, depending on the number of antibodies to assay, and the number of samples to screen. Biological samples are spotted or printed (see below) in a matrix arrangement within each compartment on a nitrocellulose membrane. The same matrix of clinical samples, including normal and diseased, or the same matrix of protein extracts from different cancer cell lines is printed in each chamber. Each individual compartment is then overlayed with a distinct antibody (polyclonal, monoclonal, Fab fragment, monospecific, single chain, affibodies, or any other recombinant version of conventional or combinatorial antibodies), and processed for the detection of antigen-antibody complexes.

Protein Sample Analysis:

For the purpose of the invention, protein samples analyzed by MPAT may derive from fresh and frozen tissues, whether normal or disease, including from patients with cancer, benign or inflammatory conditions, and normal controls. Protein samples may derive from cell cultures, cancer cell lines, and cancer cell supernatants, and even from microdissected cell types or from a given subcellular compartment. Protein samples may also derive from patient sera or any other patient biological fluid, and prepared as described in the Examples below.

Printing of Total Protein Extracts:

Individual protein sample extracts can either be deposited and spotted manually or printed with a robotic system (Genomic Solutions Flexys, PBA Robotics, UK). Routinely equal protein amounts (250 nl of a 1 mg/ml stock solution of cancer cell line protein extract) of each sample are printed in a matrix format on the MPAT membrane, in duplicate or triplicate whenever deemed appropriate.

The membrane is then incubated for 30 min in 2% H₂O₂ (hydrogen peroxide) solution to inhibit endogenous peroxidase present in the clinical samples, rinsed twice in Tris-saline buffer (TNE: 10 Tris-HCl pH 7.5, 50 mM NaCl, 2.5 mM EDTA) and then blocked for 30 min with a solution of 1% non-fat dry milk in Tris-saline buffer containing 0.1% (w/v) Tween 20 (TNET).

Antibodies:

Subsequently, each chamber or each sample matrix is overlaid with a given primary antibody. Routinely antibodies are diluted appropriately in blocking solution, followed by 1 hr incubation at RT with constant shaking. Blocking solution is TNET containing 1% non-fat dry milk or equivalent blocking solutions.

Detection of Antigen-Antibody Complexes:

The membrane is washed 5 times for 5 min each in TNET, then incubated for 1 hr with secondary antibodies conjugated with horseradish peroxidase (Roche) diluted 1:10,000 in blocking solution. The membrane is then further washed 5 times as described previously. The antigen-antibody-anti-antibody complex reactivity is measured by chemiluminescence, using the SuperSignal West Dura Extended Duration Substrate (Pierce). The image is captured using a CCD-camera (charge-coupled device; UVP model Biochemi, CCD camera grade 0, with dark room designed for chemiluminescence, fluorescence and visible).

Alternatively, instead of a chemiluminescent-based detection and a CCD-camera based image acquisition system, a fluorescent-based system can be used, incorporating for example the use of the Li-cor Odyssey infrared imaging acquisition system. The MPAT protocol is then modified accordingly. Peroxidase inhibition is not necessary. The membrane is rinsed twice in Tris-saline buffer, and then blocked for 30 min in Odyssey blocking solution (Li-Cor). Primary antibody is appropriately diluted in Odyssey blocking solution, followed by 1 hr incubation at RT. The membrane is washed 5 times for 5 min each in TNET, then incubated for 1 hr with secondary antibodies labeled with a fluoresecent dye (IgG-IRDye 800CW) diluted 1:10,000 in Odyssey blocking solution. The membrane is then further washed 5 times as described previously. The antigen-antibody-anti-antibody complex is measured by direct infrared fluorescence detection. The intensity of each complex is captured as an image by scanning the membrane with Odyssey infrared imaging system in the 800 nm channel at 84 m resolution. Protocols based on different labeling and detection systems, such as alkaline-phosphatase, biotin-streptavidine, and fluorophores as described can also be successfully performed within the scope of the present invention.

The following internal controls can be routinely provided: i) the same matrix of samples is overlaid with buffer rather than with primary antibody, followed by the secondary antibody, thus revealing the background of the secondary antibody (no antibody control); and ii) the same matrix of samples is overlaid with pre-immune serum, or non-secreting hybridoma or dilution buffer, followed by secondary antibody, thus revealing the nonspecific binding of mouse immunoglobulins.

### Example 4

### BF819 Immunodetection by Matrix Protein Array in Patient Tissues

Clinical Samples:

Frozen human tissue biopsies with annotated pathology report have been acquired from the Cooperative Human Tissue Network (CHTN). All specimens are tissue samples collected prior to any treatment. Specimens are provided with corresponding pathology report and well-annotated clinical information (disease condition, cancer histological type, clinical history, stage, age, gender, race; Jewell, 2002). Clinical samples are stored in −70° C. freezers, and immediately prior to assay, samples are aliquoted in ice to avoid multiple freeze-thaw cycles, and the original tube is maintained at −70° C. The collection amounts to ˜1,500 cancer tissues covering all major malignancies, comprising colorectal cancer, lung, pancreatic and prostate cancers, melanoma, renal carcinoma, and gynecological cancers, including breast, ovarian, uterine, and cervical cancers.

Up to five independent studies addressing the differential expression of the Bm of the present invention in cancer versus normal were performed using four different sets of clinical specimens comprising 213, 741, 1329 and 1471 tissue samples, respectively. Table 1 summarizes the composition of those sets: it provides for each relevant organ site, the total number and type of specimens, the number of normal, benign and cancer samples, including early and late stage patients.

The 213 tissue sample experiment included: 32 breast cancer, 16 breast normal and 7 benign; 64 colon cancer, 26 colon normal and NAT, and 20 benign; 32 lung cancer and 16 lung NAT, amounting to 128 cancer, 58 normal and NAT, and 27 benign samples (Table 1).

The 741 tissue sample experiment included: 115 breast cancer, and 175 breast NAT and 15 benign; 173 colon cancer, and 240 colon NAT and 23 benign, amounting to 288 cancer, 415 NAT and 38 benign samples (Table 1).

The 1329 tissue sample experiment included the following samples: 115 breast cancer, 175 breast NAT and 15 benign; 173 colon cancer, 240 colon NAT and 23 benign, 199 lung cancer, 208 lung NAT and 15 benign; 88 ovarian cancer, 43 ovarian NAT and 35 benign, amounting to 575 cancer, 666 NAT and 88 benign samples (Table 1).

The 1471 tissue samples experiment included the following samples: 116 breast cancer, 138 breast normal and NAT, and 22 benign; 198 colon cancer, 284 colon normal and NAT, and 17 benign; 186 lung cancer, 233 lung normal and NAT, and 14 benign; 123 ovarian cancer, 78 ovarian normal and NAT, and 62 benign, amounting to a total of 623 cancers, 733 normal and NAT, and 115 benign samples (Table 1).

Protein Extraction from Frozen Tissues:

Fresh or frozen tissue of human origin for the purpose of this invention, is cut off in small pieces, grounded, homogenized in a 50 mM Tris-HCl pH 7.5, 2 mM EDTA, 100 mM NaCl, 1% NP40, and 1 mM vanadate solution containing the following protease inhibitors: PMSF, aprotinin, leupeptin at 1, 2 and 4 mM respectively. The homogenate is kept on ice for 20 min and centrifuged at 14,000 rpm for 15 min. Supernatant is transferred to a new container and the tissue pellet is resuspended, and again kept on ice for 20 min and centrifuged as indicated above. Supernatant is removed and added to the first one. Protein concentration is determined according to standard conditions as known to those skilled in the art. Protein solution is stored in a −80° C. freezer until further usage.

MPAT:

Protein extracts from frozen tissues are spotted on the MPAT and processed as described in Example 3 using mAb BF819.

### Example 5

### Immunodetection of BF819 in Cancer Cell Lines

Cancer Cell Lines:

mAb BF819 are used to detect (via MPAT) the expression of the corresponding marker in cancer cell lines deriving from a variety of cancers. In one experiment, the following 28 cancer cell lines were used: HBC4, T-47D, MDA-MB 231, and MCF7 (breast cancer); Caco-2, DLD-1, WiDr, and COLO 320DM (colon cancer); HUH7 and HePG2 (hepatic cell carcinoma); NCI-H1155, NCI-H460, NCI-H1792, SKLU-1, SK-MES, and NCI-H157 (lung cancer); RPMI 7951, M21, and FEM (melanoma); LAPC-4, PC-3, DU 145, LNCaP (prostate cancer); Raji (lymphoma); TK6 (human B lymphoblast); GBM 8401 (brain glioblastoma multiforme); KB (oropharyngeal epidermoid carcinoma); and ES2 (ovarian cancer). In another experiment, the following 4 cervical cancer cell lines were used: HPV-containing Ca Ski, ME-180, and SiHa, and the non-HPV containing C-33A. All cell lines are cultured in media according to ATCC or provider's recommendations.

Preparation of Protein Extracts from Cancer Cell Lines:

Cancer cell lines (about 10⁷) are grown in culture as recommended by ATCC provider, with 10% fetal calf serum, 100 μg/ml streptomycin and penicillin until 80% confluency, harvested, washed twice with PBS, resuspended in phosphate buffer (pH 8.0) and disrupted in the following buffer: 50 mM Tris-HCl pH 7.5, 2 mM EDTA, 100 mM NaCl, 1% NP40, and 1 mM vanadate solution containing the following protease inhibitors: PMSF, aprotinin, leupeptin at 1, 2 and 4 mM respectively. The cell lysate is centrifuged for 5 min at 14,000 rpm. Protein concentration of cancer cell extracts is determined using the BCA (bicinchoninic acid) Protein Assay Reagent Kit (Pierce, Rockford, Ill.) using a 1:200 dilution of extract, and a BSA standard. A microplate reader (vmax, Molecular Device) is used to read the absorbance at 570 nm. Stock solutions of protein extracts at 1 mg/ml are used.

MPAT:

Protein extracts from cancer cell lines are spotted on the MPAT in duplicate and processed as described in Example 3 using mAb BF819.

### Example 6

### Staining of Cancer Tissues by Immunohistochemistry

To demonstrate the specificity of the marker and monoclonal antibody of the present invention, and their use in histology-based diagnostic applications, tissue slides or tissue arrays displaying tissues from cancer and benign patient, and normal controls (matched, i.e. from the same patient, or unmatched) are used as follows.

5-μm formalin-fixed paraffin-embedded human tissue section slides or tissue microarrays are deparaffinized by baking slides in oven at 60° C. for 30 min followed by immersion in three xylene baths for 5 min each. Slides are rehydrated by immersion in two 100% ethanol baths for 5 min each, then in 95% ethanol, 70% ethanol baths for 3 min each, and finally soaked in water.

Endogenous peroxidase is blocked by treating slides with 3% hydrogen peroxide solution in PBS for 10 min at RT, then washing them twice in PBS for 3 min each. Antigen retrieval is obtained by heating slides in a pressure cooker at full pressure for 5 min in 10 mM Tris, 1 mM EDTA pH 9, or in Tris-sodium citrate 10 mM, 0.05% Tween 20 pH 6. Slides are then cooled to RT in the same buffer for 10-20 min, rinsed in tap water for 3 min, and finally immersed in Tris buffer.

To block endogenous biotin, which may be a problem in some tissues, slides are incubated for 15 min at RT in a streptavidin solution in PBS (100 μg/ml), rinsed with Tris buffer, followed by incubation with biotin solution (500 μg/ml) in PBE (PBS with 1% BSA, 1 mM EDTA, 1.5 mM NaN₃ pH 7.4) for 30-60 min at RT, and washed in PBS. Non-specific binding is further blocked by treating slides for 15 min at RT in 3% horse serum diluted in PBE.

Slides are incubated with mAb BF819 (either undiluted cell culture supernatant, or appropriately diluted 1:2-1:20 in PBE buffer) for 30 min at 37° C., or 1 hr at RT or overnight at 4° C. in a humidity chamber, then rinsed 3 times for 5 min each in Tris buffer. Slides are covered with a 1:1000 dilution of biotinylated secondary antibody in PBE buffer, and incubated for 30 min at 37° C. or 1 hr at RT, then washed 3 times for 5 min each in Tris buffer. Slides are then covered with 1:1000 dilution of peroxidase-conjugated streptavidin diluted in PBE (without azide), and incubated for 30 min at 37° C. or 1 hr at RT, then washed 3 times for 5 min each in Tris buffer.

Finally, a few drops of AEC substrate solution (1 ml of 4 mg/ml AEC stock solution in DMF, plus 15 ml of 0.1 M Na acetate pH 5, and 15 μl of 30% hydrogen peroxide) are used to cover the slides. The reaction is allowed to pursue for 10-40 min, then visualized under the microscope, and stopped with tap water whenever appropriate. Slides are rinsed in water, and counterstained with a few drops of weak Mayer's hematoxylin solution for 1-2 min. Slides are then immersed in 0.1% sodium bicarbonate solution until nuclei turn blue. Slides are covered with aqueous mount media, placed in an oven at 70° C. and then let dry for 10-20 min or overnight at RT.

### Example 7

### Western Blot Analysis

Total protein extracts (equivalent to 10 microgram per lane) from a given tissue or cancer cell extract are loaded, separated on polyacrylamide-SDS and transferred onto nitrocellulose membrane according to standard procedures. Different percentage of polyacrylamide may be used as known by those skilled in the art, depending on the expected molecular weight of the marker, and nitrocellulose can be replaced by PVDF, nylon membrane or other support. After transfer, the membrane is saturated for 1 hr in TNE/Tween blocking buffer (10 mM Tris-HCl pH 7.5, 2.5 mM EDTA, 50 mM NaCl, 0.1% Tween 20) containing 2.5% dried non-fat milk. The membrane is used as is or cut into strips of different size as necessary. Each membrane section or strip is first blocked with BSA or other commonly used blocking agent, then incubated for 1 hr with an antibody at appropriate dilution in the blocking buffer.

The blot is washed 5 times in the same buffer described above and incubated for 1 hr with a goat anti-mouse secondary antibody conjugated with IRDye 800CW fluorescent dye (Li-cor) according to manufacturer's instructions. Then membrane sections or strips are washed 5 times for 10 min each in TNE/Tween without milk. Antigen-antibody complexes are visualized by scanning the membrane sections or strips using the Odyssey infrared Imaging System (Li-cor) according to manufacturer's instructions. Other detection systems, known to the skilled in the art, can be used as well.

### Example 8

### Detection of Secreted Bm from Cancer Cell Lines

Cancer Cell Lines:

To test for the presence of secreted BF819 in cancer cell lines, cell culture supernatants were assayed with mAb BF819 of the present invention via MPAT. In one experiment, the following 12 cancer cell lines were used: NCI-H157, NCI-H1155, NCI-H838, A549, NCI-H1792 (lung cancer); DU 145 and PC-3 (prostate cancer); ES2 (ovarian cancer); MCF7 and MDA-MB 231 (breast cancer); COLO 320DM (colon cancer); NEF, normal embryonic fibroblasts as normal cell line control. In another experiment, the following 4 cervical cancer cell lines were used: HPV-containing Ca Ski, ME-180, and SiHa, and the non-HPV containing C-33A.

Preparation of Cell Culture Supernatants:

Tissue culture supernatants (TCS) are centrifuged to remove cell debris and supernatants are precipitated by slow addition of 1-1.5 volumes of ice-cold acetone. Precipitation is carried out on ice or at −20° C. for 1 hr. After 15 min centrifugation at 4° C. using pre-cooled rotors, tubes are inverted to completely remove supernatants. Pellets are quickly recentrifuged to fully eliminate the last drops of supernatant. Finally pellets are allowed to dry for 5-10 min under the hood, and resuspended in 2.5 ml of Tris 50 mM pH 7. Samples are homogenized with sonicator, whenever needed, and protein concentration is measured via a BCA assay (see above). Samples are diluted to 1 mg/ml working solutions.

To prepare TCS without fetal calf serum (FCS) to facilitate analysis of potentially secreted proteins by immunodetection analysis of cancer cell lines by MPAT, cells are grown to 70% confluency, complete medium is removed and replaced with medium without fetal calf serum (FCS) and grown for 25 hr at 37° C. Cell culture supernatant without FCS is then precipitated as above.

MPAT:

Protein extracts from cell culture supernatants are spotted on the MPAT in duplicate and processed as described in Example 3 using mAb BF819.

### Example 9

### Detection of Secreted Marker BF819 in Serum

Clinical Samples:

All specimens are pre-operative serum samples collected prior to any treatment, and acquired from the Cooperative Human Tissue Network (CHTN) with pathology report and well-annotated clinical information (disease condition, cancer histological type, clinical history, stage, age, gender, race; Jewell, 2002). Clinical samples are stored in −70 C freezers, and immediately prior to assay, samples are aliquoted in ice to avoid multiple freeze-thaw cycles, and the original tube is maintained at −70 C. From a collection of over 2,000 cancer serum samples covering all major malignancies, the serum sample set used to test for the presence of the Bm of the present invention in patient serum comprised 165 samples as follows: 91 samples from pancreatic cancer patients (50 early, 34 late and 7 of unknown stage), 16 from benign pancreatic tumor patients and 14 from pancreatitis patients, and 44 samples as normal controls (Table 2).

Preparation of Serum Samples:

Serum samples are diluted 1:33 in Tris-saline buffer containing a protease inhibitor cocktail (leupeptine, aprotinin, PMSF and soybean trypsin inhibitor).

MPAT:

Serum samples (250 nanoliters of a 1:33 working solution) are spotted on the MPAT in duplicate and processed as described in Example 3 using the mAb of the present invention, and the fluorescence-based Li-cor Odyssey detection system.

### Example 10

### Detection of Secreted Marker BF819 in Urine

Clinical Samples:

The clinical sample set for the “precipitated” urine sample experiment comprised 305 clinical samples from a variety of cancer patients, benign and normal controls, comprising (Table 4): 35 colon cancer cases (including 12 stage I, 10 stage II, 12 stage III, 1 stage IV); 32 cases of inflammatory conditions of the colon (including 14 chronic colitis, 8 diverticulitis, and 10 Crohns disease); 10 cases of benign colon disease; 6 kidney cancers patients (including 3 stage I, 1 stage II, and 2 stage III); 1 kidney benign; 21 pancreatic cancer (mostly early stage, including 3 stage I, 15 stage II, 1 late and 2 unknown), and 1 benign pancreatic tumor; 107 prostate cancer patients (including 55 stage II, 45 stage III, and 7 stage IV) and 92 normal controls. Note that cancer stages I and II are defined as “early”, while stages III and IV are defined as “late”.

The clinical samples set for the “unprecipitated” (as is) urine sample experiment comprised 47 samples as follows: 10 colon cancer patients (6 stage I, 4 stage II), 5 with inflammatory conditions of the colon, 4 with benign conditions of the colon, 13 prostate cancer patients (11 stage II, and 2 stage III), 5 pancreatic cancer patients (4 stage II and 1 unknown), and 10 normal controls (Table 4).

Preparation of Urine Samples:

To prepare “precipitated” urine samples, total proteins were precipitated by 20-50% v/v ice-cold acetone. Briefly, urine samples are centrifuged to remove debris. Supernatants are transferred to fresh tubes and precipitated by the slow addition of 1 volume of ice-cold acetone (20-50%). Precipitation is carried out on ice or at −20° C. for 1 hr. After 15 min centrifugation at 4° C. using pre-cooled rotors at 3,000 g, tubes are inverted to completely remove supernatants. Pellets are quickly recentrifuged to fully eliminate the last drops of supernatant. Finally, pellets are allowed to air dry for 5-10 min under the hood, and resuspended in 2.5 ml of Tris-Triton buffer. Samples are homogenized on ice with sonicator, whenever needed, and protein concentration is measured via a BCA assay (see above). Protein concentration is then adjusted to yield a 0.3 mg/ml working solution, and 250 nanoliters of the latter are printed on the MPAT membrane.

Unprecipitated urine samples are first centrifuged to remove debris whenever turbid, then 250 nanoliters of each urine sample are printed on the MPAT either “as is” or upon dilution 1:2 or 1:10 in Tris-Triton buffer.

MPAT:

After spotting on the MPAT membrane, samples are probed with mAb BF819 by incubation for 30 min, followed by six washes, processed and visualized as described in Example 3.

An assessment of BF819 in an individual can be translated to an assessment of cancer for the individual, including a score or other identifier that indicates whether an individual has cancer or that indicates a certain likelihood that the individual has cancer or that identifies additional known markers for disease initiation, progression, metastasis or any other characteristic of neoplastic disorders. Similarly, the score or other identifier may indicate a specific type of cancer assessment, such as the assessments of various cancer characteristics described herein, including (but not limited to), determination of whether an individual's cancer is metastasized, determination of the stage of an individual's cancer (such as distinguishing between stage I and stage Ill cancer), determination of whether an individual's cancer is SCLC or NSCLC determining whether a lung lesion identified in an individual (such as by CT screening) is a malignant tumor or a benign lesion, and determining tumor regression and/or recurrence.

As noted above, the invention includes methods for diagnosing diseases having differential expression of BF819. For example, normal, control, or standard values (e.g., that represent typical expression levels of a protein in healthy individuals) for BF819 can be established in various assay formats, such as by combining body fluids, tissues, or cell extracts taken from a patient with specific antibodies to a protein under conditions for complex formation. Standard values for complex formation in normal and disease tissues can be established by various methods, such as photometric means. Complex formation, as it is expressed in a test sample, can be compared with the standard values for correlation to disease. Deviation from a normal standard and toward a disease standard can provide parameters for disease diagnosis or prognosis while deviation away from a disease standard and toward a normal standard can be used to evaluate treatment efficacy. Alternately, threshold levels of disease or normal are established.

Platform immunological methods for detecting and measuring complex formation as a measure of BF819 expression using either specific polyclonal or monoclonal antibodies are known in the art. Examples of such techniques include ELISAs, radio-immunoassays (Ms), flow cytometry (also referred to as fluorescence-activated cell sorting, or FACS), and antibody arrays.

For example, ELISA can be used to detect or quantify BF819. In certain exemplary ELISA methods, an antibody that specifically binds BF819 may be coated to the well of a suitable container (e.g., a 96 well microliter plate), a patient sample (e.g., a serum sample) can be added to the well and incubated for a period of time, and the presence of BF819 in the patient sample can be detected upon binding of an epitope on a BF819 polypeptide in the patient sample to the antibody that is coated to the well. In this instance, a second antibody conjugated to a detectable moiety may optionally be added following the addition of the patient sample to the coated well. ELISA methods such as these may be modified or optimized as desired.

Further, instead of coating the well with the BF819 mAb, BF819 may be coated to the well. Thus, in certain ELISA methods, a BF819 polypeptide is coated to the well of a suitable container (e.g., a 96 well microliter plate), a BF819 mAb (which may optionally be conjugated to a detectable moiety such as an enzymatic substrate (horseradish peroxidase or alkaline phosphatase)) is added to the well and incubated for a period of time, and the presence of BF819 is detected. An antibody to BF819, whether the BF819 mAb or another, does not have to be conjugated to a detectable moiety; for example, a second antibody (which recognizes the antibody to BF819 or the BF819 mAb disclosed herein) is conjugated to a detectable moeity added to the well.

These assays and their quantitation against purified, labeled standards are well known in the art (Ausubel, supra, unit 10:1-10.6). For example, a two-site, monoclonal-based immunoassay utilizing antibodies reactive to two non-interfering epitopes can be utilized, and competitive binding assay can also be utilized (Pound (1998) Immunochemical Protocols, Humana Press, Totowa N.J.).

For diagnostic applications, an antibody can be labeled with a detectable moiety (interchangeably referred to as a “label” or “detectable substance”), such as to facilitate detection by various imaging methods. Methods for detection of labels include, but are not limited to, fluorescence, light, confocal, and electron microscopy; magnetic resonance imaging and spectroscopy; fluoroscopy, computed tomography and positron emission tomography. Numerous detectable moieties are available for labeling antibodies, including, but not limited to: 1) radioisotopes, such as ³⁶S, ¹⁴C, ¹²⁵I, ³H, and ¹³¹I, 2) fluorescent labels such as rare earth chelates (europium chelates) or fluorescein and its derivatives, rhodamine and its derivatives, dansyl, Lissamine, phycoerythrin and Texas Red, 3) enzyme-substrate labels (e.g., U.S. Pat. Nos. 4,275,149 and 4,318,980).

BF819 can be detected in vivo in an individual patient by introducing into the patient a labeled antibody (or other type of detection agent) specific for the protein marker. For example, an antibody can be labeled with a radioactive marker as described above whose presence and location in an individual can be detected by standard imaging techniques.

The present invention also includes the combination of any detection of the BF819 protein, polynucleotide or the BG9844 mAb with the use of any existing marker in a combination assay system, kit or method wherein the combination of the detection or measurement of the two markers is correlated to the detection of cancer presence, progression, risk or any other parameter described herein. The existing markers include but are not limited to Ki-67 (Ki-67), P161NK4a (p16), Estrogen receptor (ER-alfa), Progesterone receptor (PR), c-erbB-2 (HER-2), Cathepsin D, CA15-3 (CA15-3), CA27.29 (CA27.29), Carcinoma embryonic antigen (CEA), Vimentin (Vimentin), Prostate specific antigen (PSA), Prostatic acid phosphatase (PAP), Kallikrein-2 (KLK-2), p504S (p504S), Tumor Protein p63 (p63), Chromogranin A (CgA), Progastrin releasing peptide type 3 (ProGRP), Neuron specific enolase (NSE), Melanocyte lineage-specific antigen (Gp100), MART-1 (MART-1), MAGE-1 (MAGE-1), Calcium binding protein A4/Metastasin 100 (S100A4), Alfa-fetoprotein (AFP), Macrophage inhibitory cytokine (MIC-1), Osteopontin (OSPN), CA19-9 (CA19-9), Mucin-16/ovarian carcinoma antigen CA-125 (CA-125), Leukocyte common antigen (CD45 LCA), CD68 (CD68), Cytokeratins 5, 6 (CK5/6), Cytokeratin 16, 17 and 18 (CK16/17/18), Cytokeratin 17 (CK17), Cytokeratin 19 fragment/CYFRA 21.1, B-cell lymphoma-2 (BCL-2), B-Lymphocyte antigen (CD20), Hematopoietic progenitor CD34 (CD34), Proto-oncogene P53 (p53), Mucin-2 (MUC-2), Mucin-3A (MUC-3), Mucin-4 (MUC-4), Mucin 5AC (MUC-5AC), Mucin-6 (MUC-6), Proliferating cell nuclear antigen (PCNA), Tyrosinase (Tyr), Prostate specific membrane antigen (PSMA-1), Calcium binding protein (S1002), Tissue inhibitor of metalloproteinase (TIMP-1), Squamous cell carcinoma antigen (SCC), Androgen Receptor (ARC), Urokinase plasminogen activator (UPA), Plasminogen activator inhibitor (PAI), Protein uncharacterized ENSP0381381, CA-242, CYFRA21-1.

A description of the use and potential applicability of the markers to the present invention is provided at the following which are incorporated by reference. Duffy M J, Esteva F J, Harbeck N, Hayes D F, Molina R. Tumor markers in breast cancer, In: National Academy of Clinical Biochemistry (NACB) Laboratory Medicine Practice Guidelines “Use of Tumor Markers in testicular, prostate, colorectal, breast and ovarian cancer”, Sturgeon C M, Diamandis E P, Ed., Chapter 5, pp 37-49, 2009; Harris L et al., American Society of Clinical Oncology, Update of Recommendations for the Use of Tumor Markers in Breast Cancer, J Clin Oncol 25 (33): 5287-5312, 2007; see also for Goggins M, Koopmann J, Yang D, Canto M I, Hruban R H. National Academy of Clinical Biochemistry (NACB) Laboratory Medicine Practice Guidelines for the Use of Tumor Markers in Pancreatic Ductal Adenocarcinoma. www.nacb.org/tumors, 2005; see also for Brunner N, Duffy M J, Haglund C, Holten-Andersen M, Nielsen H J. Tumor markers in colorectal malignancy, In: National Academy of Clinical Biochemistry (NACB) Laboratory Medicine Practice Guidelines “Use of Tumor Markers in testicular, prostate, colorectal, breast and ovarian cancer”, Sturgeon C M, Diamandis E P, Ed., Chapter 4, pp 27-35, 2009; Locker G Y, Hamilton S, Harris J, Jessup J M, Kemeny N, Macdonald J S, Somerfield M R, Hayes D F, Bast R C Jr., ASCO Tumor Panel Expert Panel. ASCO 2006 update of recommendations for the use of tumor markers in gastrointestinal cancer. J Oncol, November 20; 24(33):5313-27, 2006; see also for Stieber P, Hatz R, Molina R, von Pawel J, Schalhorn A, Schneider J, Yamaguchi K. Tumor markers in lung cancer, In: National Academy of Clinical Biochemistry (NACB) Laboratory Medicine Practice Guidelines “Use of Tumor Markers in testicular, prostate, colorectal, breast and ovarian cancer”, Sturgeon C M, Diamandis E P, Ed., 2006; see also for Chan D W, Bast R C Jr, Shih I-M. Sokoll L, Soletormos G. Tumor markers in ovarian cancer, In: National Academy of Clinical Biochemistry (NACB) Laboratory Medicine Practice Guidelines “Use of Tumor Markers in testicular, prostate, colorectal, breast and ovarian cancer”, Sturgeon C M, Diamandis E P, Ed., Chapter 6, pp 51-59, 2009; see also for Lilja H, Semjonow A, Sibley P, Babaian R, Dowell B, Rittenhouse H, Sokoll L. R. Tumor markers in prostate cancer, In: National Academy of Clinical Biochemistry (NACB) Laboratory Medicine Practice Guidelines “Use of Tumor Markers in testicular, prostate, colorectal, breast and ovarian cancer”, Sturgeon C M, Diamandis E P, Ed., Chapter 3, pp 15-25, 2009.

BF819 assays are provided that have at least 70% sensitivity at 95% specificity, or at least 70% specificity at 95% sensitivity. In certain embodiments, BF819 assays are provided that have at least 85% sensitivity at 95% specificity, or at least 85% specificity at 95% sensitivity. In further embodiments, BF819 assays are provided that have at least 90% sensitivity or at least 90% specificity, or that have at least 95% sensitivity or at least 95% specificity. In yet further embodiments, assays are provided that have at least 70, 75, 80, 85, 90, 91, 92, 93, 94, 95, 96, 97, 98, or 99% (or any other percentage in-between) sensitivity and 70, 75, 80, 85, 90, 91, 92, 93, 94, 95, 96, 97, 98, or 99% (or any other percentage in-between) specificity. In yet further embodiments, BF819 assays are provided that have at least 0.7, 0.75, 0.8, 0.85, 0.9, 0.91, 0.92, 0.93, 0.94, 0.95, 0.96, 0.97, 0.98, or 0.99 (or any other value in-between).

Assay devices for detection of BF819 can be provided in the form of kits, such as for use in performing the methods disclosed herein. Furthermore, any kit can contain one or more detectable labels (e.g., detactably labeled reagents such as antibodies), such as a fluorescent moiety, etc. A kit can comprise (a) reagents comprising at least one antibody for detecting BF819, and optionally (b) known markers specific for cancer or a type of cancer of interest.

For immunohistochemistry, a disease tissue sample may be, for example, fresh or frozen or may be embedded in paraffin and fixed with a preservative such as formalin. A fixed or embedded section can be contacted with a labeled primary antibody to BF819 and secondary antibody, wherein the antibody is used to detect BF819 protein expression in situ.

Antibodies can be used to detect BF819 in situ, in vitro, or in a cell lysate or supernatant in order to evaluate the abundance and pattern of expression. Also, antibodies can be used to assess abnormal tissue distribution or abnormal expression during development or progression of a biological condition. Antibodies against BF819 are useful for detecting the presence of the proteins in cells or tissues to determine the pattern of expression of the proteins among various tissues in an organism and over the course of the organism's development.

Further, mAb BF819 is used to assess expression in disease states such as in active stages of a disease or in an individual with a predisposition toward disease related to the protein's function. When a disorder is caused by inappropriate tissue distribution, developmental expression, or level of expression of BF819, an antibody can be prepared against the normal protein. If a disorder is characterized by a specific mutation in a BF819 protein, antibodies specific for the mutant protein can be used to assay for the presence of the specific mutant.

In certain embodiments, the invention provides detection or diagnostic methods of BF819 using LC/MS. The differential expression of BF819 in disease and healthy (or drug-resistant and drug-sensitive, for example) samples can be quantitated using mass spectrometry and ICAT (Isotope Coded Affinity Tag) labeling, which is known in the art. ICAT is an isotope label technique that allows for discrimination between two populations of proteins, such as a healthy and a disease sample. Overexpression or under-expression of BF819, as measured by ICAT, can indicate, for example, the likelihood of having or developing a disease or an associated pathology.

LC/MS spectra can be correlated to disease and normal samples and processed as follows. The raw scans from the LC/MS instrument can be individualized for peak detection and to isolate sequence information using signal/noise reduction software. Filtered peak lists can then be used to detect ‘features’ corresponding to specific BF819 polypeptides from the original sample(s). Features are characterized by their mass/charge ratio, charge, intensity, retention time, isotope pattern, sequence, for example through labeled residue sequencing to determine examples of the sequence (SEQ ID NO: 1) of BF819 to separate disease from normal.

The signal intensity BF819 present in both healthy and disease samples can be used to calculate the differential expression, or relative abundance, of the polypeptide. The intensity of a peptide found exclusively in one sample can be used to calculate a theoretical expression ratio for that peptide. Expression ratios can be calculated for each peptide in an assay or experiment.

Natural or synthetic polynucleotides are useful as hybridization probes for determining the presence, level, form, and/or distribution of BF819 nucleic acid expression. Exemplary probes can be used to detect the presence of, or to determine levels of, a specific nucleic acid molecule in cells, tissues, and in organisms. Accordingly, probes corresponding to BF819 as described herein can be used to assess expression and/or gene copy number in a given patient sample, cell, tissue, or organism, which can be applied to, for example, diagnosis of disorders involving an increase or decrease in BF819 protein expression relative to normal BF819 protein expression levels.

Nucleic acid test kits for detecting the presence of natural BF819 polynucleotides (e.g., mRNA or genomic DNA) in a biological sample comprise reagents such as a labeled or labelable nucleic acid or agent capable of detecting BF819 polynucleotide in a biological sample and means for comparing the amount of BF819 polynucleotide in the sample with a standard.

Detection of mutations such as deletions, additions, or substitutions of one or more nucleotides in a gene, chromosomal rearrangements (such as inversions or transpositions), and modification of genomic DNA such as aberrant methylation patterns or changes in gene copy number or amplification can be detected at the nucleic acid level by a variety of techniques. For example, genomic DNA or RNA from a patient or group of patients can be analyzed directly or can be amplified (e.g., using PCR) prior to analysis. In certain exemplary embodiments, detection of a mutation involves the use of a probe/primer in a PCR reaction (see, e.g. U.S. Pat. Nos. 4,683,195 and 4,683,202), such as anchor PCR or RACE PCR, or, alternatively, in a ligation chain reaction (LCR) (see, e.g., Landegran et al., Science 241:1077-1080 (1988) and Nakazawa et al., PNAS 91:360-364 (1994)), the latter of which can be particularly useful for detecting point mutations in a gene (see Abravaya et al., Nucleic Acids Res. 23:675-682 (1995)). Exemplary methods such as these can include the steps of collecting a biological sample from a patient, isolating nucleic acid (e.g., genomic, mRNA, or both) from the cells of the sample, contacting the nucleic acid with one or more primers which specifically hybridize to a marker nucleic acid under conditions such that hybridization and amplification of the marker nucleic acid (if present) occurs, and detecting the presence or absence of an amplification product, or defecting the size of the amplification product and comparing the length to a control sample. Deletions and insertions can be detected by a change in size of the amplified product compared to a normal genotype. Point mutations can be identified by hybridizing amplified DNA to normal RNA or antisense DNA sequences, for example.

Alternatively, mutations in BF819 polynucleotide can be identified, for example, by alterations in restriction enzyme digestion patterns as determined by gel electrophoresis. Further, sequence-specific ribozymes (U.S. Pat. No. 5,498,531) can be used to identify the presence of specific mutations by development or loss of a ribozyme cleavage site. Perfectly matched sequences can be distinguished from mismatched sequences by nuclease cleavage digestion assays or by differences in melting temperature.

Sequence changes at specific locations can be assessed by nuclease protection assays such as RNase or chemical cleavage methods. Furthermore, sequence differences between a mutant BF819 gene and a corresponding wild-type gene can be determined by direct DNA sequencing. A variety of automated sequencing procedures can be utilized when performing diagnostic assays (Naeve, C. W., (1995) Biotechniques 19:448), including sequencing by mass spectrometry (e.g., PCT International Publication No. WO 94/16101; Cohen et al., Adv. Chromatogr. 36:127-162 (1996); and Griffin et al., Appl. Biochem. Biotechnol. 38:147-159 (1993)).

Methods for detecting mutations in a BF819 polynucleotide also include methods in which protection from cleavage agents is used to detect mismatched bases in RNA/RNA or RNA/DNA duplexes (Myers et al., Science 230:1242 (1985)); Cotton et al., q/b PNAS 85:4397 (1988); Saleeba et al., Meth. Enzmmol. 217:286-295 (1992)), electrophoretic mobility of mutant and wild type nucleic acid is compared (Orita et al., q/b PNAS 86:2766 (1989); Cotton et al., Maw. Res. 285:125-144 (1993); and Hayashi et al., Genet. Anal. Tech. Appl. 9:73-79 (1992)), and movement of mutant or wild-type fragments in polyacrylamide gels containing a gradient of denaturant is assayed using denaturing gradient gel electrophoresis (DGGE) (Myers et al., Nature 313:495 (1985)). Examples of other techniques for detecting point mutations include selective oligonucleotide hybridization, selective amplification, and selective primer extension.

Natural and synthetic molecules of the invention are also useful for monitoring the effectiveness of modulating agents on the expression or activity of BF819, such as in clinical trials or in a treatment regimen. For example, the gene expression pattern of a BF819 natural polynucleotide expression or the presence or amounts of the BF819 marker can serve as a barometer for the continuing effectiveness of treatment. The gene expression pattern can also serve as a marker indicative of a physiological response such as resistance or sensitivity of the cancer cells to the compound. For example, based on monitoring nucleic acid expression, the administration of a compound can be increased or alternative compounds to which the patient has not become resistant can be administered.

In one embodiment, the level of BF819 mRNA is determined either by in situ and by in vitro formats in a biological sample using methods known in the art. Many expression detection methods use isolated RNA. For in vitro methods, any RNA isolation technique that does not select against the isolation of mRNA can be utilized for the purification of RNA from tumor, tissue samples, or tissue cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The mRNA is used in hybridization or amplification assays that include, but are not limited to, Southern or Northern analyses, polymerase chain reaction analyses and probe arrays. One preferred diagnostic method for the detection of mRNA levels involves contacting the mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of BF819 mRNA with the probe indicates that BF819 is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods of several formats having probes linked to a variety of detection systems (such as radioactive or fluorescent probes) for use in detecting the level of BF819.

For in situ methods, BF819 mRNA need not be isolated from the tissue or tumor cells prior to detection. In such methods, a cell or tissue sample is prepared/processed using known histological methods. The sample is then immobilized on a support, typically a glass slide, and then contacted with a probe that can hybridize to BF819 mRNA.

The invention also includes vectors and host cells containing natural and synthetic BF819 nucleic acid molecules. The term “vector” refers to a vehicle, such as a nucleic acid molecule, which can transport BF819 polynucleotides. When the vector is a nucleic acid molecule, the BF819 polynucleotides are covalently linked to the vector nucleic acid to yield a synthetic polynucleotide. A vector can be, for example, a plasmid, single or double stranded phage, a single or double stranded RNA or DNA viral vector, a mini-locus or artificial chromosome, such as a BAC, PAC, YAC, or MAC. A vector can be maintained in a host cell as an extrachromosomal element such as a plasmid where it replicates and produces additional copies of BF819 polynucleotides or the vector can integrate into the host cell genome and produce additional copies of BF819 polynucleotides when the host cell replicates.

Vectors of the invention include maintenance (cloning vectors) and vectors for expression (expression vectors) of the nucleic acid molecules, for example. Expression vectors can express a portion of, or all of, a protein sequence. Vectors can function in prokaryotic or eukaryotic cells or in both (shuttle vectors). Vectors also include insertion vectors, which integrate a nucleic acid molecule into another nucleic acid molecule, such as into the cellular genome (such as to alter in situ expression of a gene and/or gene product). For example, an endogenous protein-coding sequence can be entirely or partially replaced via homologous recombination with a variant of the protein-coding sequence containing one or more specifically introduced mutations. Expression vectors can contain cis-acting regulatory regions that are operably linked in the vector to the BF819 polynucleotide such that transcription of the polynucleotide is allowed in a host cell. BF819 polynucleotides can be introduced into the host cell with a separate nucleic acid molecule capable of affecting transcription. The separate nucleic acid molecule may provide, for example, a trans-acting factor interacting with the cis-regulatory control region to allow transcription of the nucleic acid molecules from the vector. Alternatively, a trans-acting factor may be supplied by a host cell. Additionally, a trans-acting factor can be produced from a vector itself.

Regulatory sequences to which BF819 nucleic acid molecules can be operably linked include, for example, promoters for directing mRNA transcription. These include, but are not limited to, the left promoter from T7 bacteriophage promoter, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors can also include regions that modulate transcription, such as repressor binding sites and enhancers. Examples include the SV40 enhancer, the cytomegalovirus immediate early enhancer, polyoma enhancer, adenovirus enhancers, and retrovirus enhancers.

In addition to containing sites for transcription initiation and control, expression vectors can also contain sequences necessary for transcription termination and, in the transcribed region, a ribosome binding site for translation. Other regulatory control elements for expression include translation, initiation, and termination codons as well as polyadenylation signals. Numerous regulatory sequences useful in expression vectors are well known in the art (e.g., Sambrook et al., Molecular Cloning: A Laboratory Manual. 3rd. ed., Cold Spring Harbor Laboratory Press, Cold Spring Harbor, N.Y. (2001)).

A variety of expression vectors can be used to express a BF819 polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al., Molecular Cloning: A Laboratory Manual. 3rd. ed., Cold Spring Harbor Laboratory Press, Cold Spring Harbor, N.Y. (2001). Bacterial cells include, but are not limited to, E. coli, Streptomyces, and Salmonella typhimurium. Eukaryotic cells include, but are not limited to, yeast, insect cells such as Drosophila, animal cells such as COS and CHO cells (e.g., DG44 or CHO-s), and plant cells.

A regulatory sequence can provide constitutive expression in one or more host cells (e.g., tissue specific) or can provide for inducible expression in one or more cell types such as by temperature, nutrient additive, or exogenous factors such as a hormone or other ligand. A variety of vectors providing for constitutive and inducible expression in prokaryotic and eukaryotic hosts are well known in the art.

Recombinant host cells can be prepared by introducing vector constructs, such as described herein, into cells by techniques readily available to a person of ordinary skill in the art. These techniques include, but are not limited to, calcium phosphate transfection, DEAL-dextran-mediated transfection, cationic lipid-mediated transfection, electroporation, transduction, infection, lipofection, microinjection, and other techniques such as those found in Sambrook, et al. (Molecular Cloning: A Laboratory Manual. 3rd. ed., Cold Spring Harbor laboratory Press, Cold Spring Harbor, N.Y. (2001)).

For example, using techniques such as these, a retroviral or other viral vector can be introduced into mammalian cells. Examples of mammalian cells into which a retroviral vector can be introduced include, but are not limited to, primary mammalian cultures or continuous mammalian cultures, COS and CHO cells, NIH3T3, 293 cells (ATCC #CRL 1573), and dendritic cells.

Host cells can contain more than one vector. Thus, different polynucleotide sequences can be introduced on different vectors of the same cell. Similarly, BF819 polynucleotides can be introduced either alone or with other unrelated nucleic acid molecules such as those providing trans-acting factors for expression vectors. When more than one vector is introduced into a cell, the vectors can be introduced independently, co-introduced, or joined to the nucleic acid molecule vector.

Bacteriophage and viral vectors can be introduced into cells as packaged or encapsulated virus by standard procedures for infection and transduction. Viral vectors can be replication-competent or replication-defective. If viral replication is defective, replication can occur in host cells that provide functions that complement the defects.

If secretion of BF819 from a host cell is desired, appropriate secretion signals can be incorporated into the vector harboring the expression sequence for BF819. The signal sequence can be endogenous or heterologous to the protein.

Recombinant host cells that express BF819 or a BF819 variant have a variety of uses. For example, such host cells are useful for producing BF819 variants, which can be further purified to produce desired amounts of the protein or fragments thereof. Thus, host cells containing expression vectors are useful for protein production or for conducting cell-based assays for BF819 expression.

Predictive Medicine.

The present invention pertains to the field of predictive medicine in which diagnostic assays, prognostic assays, pharmacokinetics, and pharmacogenomics are used for prognostic (predictive) purposes to identify an asymptomatic patient or patient population or to propose a course of treatment in monitoring a cancer patient undergoing testing or treatment. Accordingly, the present invention includes the process of implementing a protocol for future use of markers for monitoring the progress of a patient or group of patients following a first screening, a first diagnosis, a plurality of additional, subsequent screenings or diagnose or treatment once this marker is detected. Accordingly, a first test for BF819 is followed by a subsequent test for BF819 or another marker at a future date, including a subsequent screening or analysis of BF819 to establish a protocol for diagnosis, including imaging, or treatment including biopsy or other surgical (i.e. resection) or chemical (chemo or immunotherapy) treatment, optimally including a prescribed time interval for future diagnosis or treatment. A preferred protocol for using BF819 or mAb BF819 preferably includes a first screening using BF819 followed by additional testing to monitor expression of BF819, optionally including another marker, at prescribed time intervals to determine progress or stages from an early stage of cancer, the risk of developing cancer beyond the stage assessed at the first screening or predicting the progression of the disease beyond any prior analysis of BF819 or mAb BF819

1. A method of detecting an indicator of cancer in a patient comprising:
assayinq a patient test sample
with a monoclonal antibody specific for CTD nuclear envelope phosphatase 1 under conditions permitting a binding reaction between the CTD nuclear envelope phosphatase 1 and the monoclonal antibody wherein the presence of CTD nuclear envelope phosphatase 1 at an elevated level in comparison to a normal level from a patient with no detectable cancer indicates the presence and/or extent of cancer, wherein the, and
assayed CTD nuclear envelope phosphatase 1 is secreted in the patient test sample and identifying a tissue or organ as a source of secreted CTD nuclear envelope phosphatase 1, wherein the tissue or organ is selected from the group consisting of colon, prostate, pancreas and combinations thereof.

2. The method of claim 1, wherein CTD nuclear envelope phosphatase 1 is a polypeptide variant thereof, having at least 90% homology to SEQ ID NO: 1 and being encoded by a polynucleotide having at least 90% homology to SEQ ID NO: 2.

3. The method of claim 1, wherein the monoclonal antibody is isotype IgG.

4. The method of claim 1, wherein the assayinq step comprises transforming secreted CTD nuclear envelope phosphatase 1 into a detectable entity by binding of the antibody to an epitope comprising an amino acid sequence of SEQ ID NO: 3.

5. The method of claim 1, wherein the monoclonal antibody is further comprised of means for detecting binding between the monoclonal antibody and CTD nuclear envelope phosphatase 1.

6. The method of claim 1, wherein the patient test sample is blood or serum.

7. The method of claim 1, wherein the identifying step is further comprised of diagnosing the patient with colon or pancreatic cancer.

8. The method of claim 7, wherein the patient test sample is precipitated or unprecipitated urine.

9. The method of claim 8, wherein the identifying step is further comprised of diagnosing the patient with prostate cancer.

10. The method of claim 1, wherein the monoclonal antibody is fixed to a solid support and the reacting step is comprised of exposing the solid support to a solution comprising the patient test sample.

11. The method of claim 1, further comprising the step of performing a second assaying step to measure a difference in expression of CTD nuclear envelope phosphatase 1 in the patient over time.

12. The method of claim 1, further comprising localizing abnormal expression of CTD nuclear envelope phosphatase 1 to a cellular or intracellular structure of an organ or tissue in a patient source of the patient test sample.

13. A method to separate a group of patients into discrete populations based on abnormal expression of CTD nuclear envelope phosphatase 1 comprising:
obtaining a biological sample from a plurality of patients;
assaying a biological sample from each patient for expression of CTD nuclear envelope phosphatase 1 wherein elevated expression of CTD nuclear envelope phosphatase 1 in breast, pancreatic, colon, or prostate tissue is compared to normal expression by transforming CTD nuclear envelope phosphatase 1 in the biological sample into a detectable entity; and
assigning each patient exhibiting abnormal CTD nuclear envelope phosphatase 1 expression to a population indicated for analysis of in vivo expression of CTD nuclear envelope phosphatase 1.

14. The method of claim 13, wherein the transformation of CTD nuclear envelope phosphatase 1 into a detectable entity comprises reacting CTD nuclear envelope phosphatase 1 in the biological sample with a monoclonal antibody specific for CTD nuclear envelope phosphatase 1.

15. The method of claim 14, wherein the patient test sample is comprised of serum or blood.

16. The method of claim 14, wherein the patient test sample is comprised of precipitated or unprecipitated urine.

17. The method of claim 13, wherein the group of patients are previously diagnosed with cancer is located in a tissue selected from the group consisting of breast, colon lung, ovarian, and prostate cancers and combinations thereof and the discrete populations are cancer patients having normal or abnormal expression of CTD nuclear envelope phosphatase 1.

18. The method of claim 14, wherein the biological sample is comprised of tissue or organ selected from the group consisting of breast, colon, lung, ovary, pancreas, prostate or protein derivatives thereof.

19. The method of claim 13, further comprising a second assaying step of a second biological sample from the patient and wherein the assigning step is comprised of assigning the patient to a population with continued or elevated abnormal expression of CTD nuclear envelope phosphatase 1 based on a comparison of the assaying steps.

20. A method to detect cancer in a patient comprising:
obtaining a biological sample from the patient containing a polypeptide,
assaying for the polypeptide having an amino acid sequence of SEQ ID NO: 1 in the biological sample by a selective binding reaction between the secreted polypeptide and an anti-CTD nuclear envelope phosphatase 1 antibody capable of transforming the secreted polypeptide into a detectable entity;
detecting the presence or absence of a complex formed by a binding event between the secreted CTD nuclear envelope phosphatase 1 polypeptide and the monoclonal antibody where the formation of the complex transforms the polypeptide into the detectable entity,
recording the presence of the detectable entity as a positive indicator of cancer in the patient, or, alternatively, recording the absence of the complex as a negative indicator of cancer in the patient.

21. The method of claim 20, wherein the biological sample is a patient test sample comprised of blood or serum.

22. The method of claim 21, wherein the cancer is prostate cancer.

23. The method of claim 20, wherein the biological sample is a patient test sample comprised of precipitated or unprecipitated urine.

24. The method of claim 23, wherein the cancer is colon, or pancreatic cancer and combinations thereof.

25. The method of claim 20, wherein transforming the secreted polypeptide into the detectable entity is comprised of reacting a second antibody specific for a complex formed by the secreted polypeptide and the monoclonal antibody.

26. The method of claim 20, wherein the detectable entity is formed by the monoclonal antibody binding the polypeptide at an epitope having an amino acid sequence of SEQ ID NO: 3.

27. The method of claim 20, wherein the biological sample is comprised of a tissue or organ selected from the group consisting of breast, colon, lung, ovary, pancreas and prostate and combinations or protein derivatives thereof.

28. The method of claim 25 wherein the steps of obtaining, assaying, detecting, and recording are repeated with a second biological sample from the same patient taken at a later time.

29. The method of claim 28, further comprising the step of comparing CTD nuclear envelope phosphatase 1 expression data with an administration of a cancer therapeutic treatment delivered to the patient.

30. A method to detect cancer in a patient comprising:
exposing a patient test sample to a monoclonal antibody specific for a polypeptide having an amino acid sequence of SEQ ID NO: 1 under reaction conditions permitting a binding reaction between the monoclonal antibody and the polypeptide at an epitope having an amino acid sequence of SEQ ID NO: 3;
detecting a presence or an absence of an antibody-polypeptide complex formed by the binding reaction of the monoclonal antibody and the polypeptide at the epitope; and
correlating the presence of the complex to cancer in the patient.
